(19) United States
(12) Patent Application Publication
(10) Pub. No.: US 2017/0051099 A1
(43) Pub. Date: Feb. 23, 2017

(54) POLYMERIC MATERIALS FOR BIO-APPLICATIONS

(71) Applicants: The Brigham and Women's Hospital, Inc., Boston, MA (US); Massachusetts Institute of Technology, Cambridge, MA (US)

(72) Inventors: Angela DiCiccio, Santa Clara, CA (US); Andrew Bellinger, Wellesley, MA (US); Carlo Giovanni Traverso, Etobicoke (CA); Dean Liang Glettig, Somerville, MA (US); Tyler Grant, Cambridge, MA (US); Robert S. Langer, Newton, MA (US); Elizabeth Walton, Reading (GB)

(73) Assignees: The Brigham and Women's Hospital, Inc., Boston, MA (US); Massachusetts Institute of Technology, Cambridge, MA (US)

(21) Appl. No.: 15/307,806

(22) PCT Filed: Apr. 29, 2015
(86) PCT No.: PCT/US2015/028311

§ 371 Date: Oct. 28, 2016

Related U.S. Application Data
(60) Provisional application No. 61/985,926, filed on Apr. 29, 2014.

(51) Int. Cl. C08G 59/22; A61K 31/573; A61K 31/357; C08G 59/68; A61K 47/48; C08G 63/664; C08G 63/87; A61K 31/7048; A61K 47/34

(52) U.S. Cl. CPC C08G 59/22; A61K 31/7048; A61K 31/573; A61K 31/357; A61K 47/34; A61K 47/48215; C08G 63/664; C08G 63/87; C08G 59/686; A61K 47/48784; C08G 2210/00; C08G 2230/00

(57) ABSTRACT

A composition comprising a biodegradable polymeric material and therapeutic agent associated with the polymeric material that advantageously can provide controlled release of the therapeutic agent, while comprising little to no auxiliary materials. In some embodiments, the composition is formed by the reaction of one or more monomers in the presence of a food grade catalyst. In another embodiment, the composition comprises a polymeric material capable of undergoing thermal reconfiguration (i.e. a dynamic network). Advantageously, the compositions and materials described herein may comprise a reconfigurable polymeric material (e.g., a thermoset polymeric material) having the strength and integrity of epoxy resins, the biomedical applicability of hydrogels, and/or the moldability of vitrimers.

### FIELD OF THE INVENTION

This invention generally relates to compositions and devices comprising polymeric materials and related applications.

### BACKGROUND OF THE INVENTION

Epoxy resins are versatile polymers having excellent mechanical strength, toughness, and dimensional stability. Epoxy resins have been employed in a variety of applications, such as protective coatings, paints, adhesives and composite materials. Many epoxy resins are thermosets, which are cured to give insoluble and intractable materials. Thermoplastic epoxy resins have also been used as composites, adhesives and coatings. In contrast to thermoset resins, thermoplastics may be modified post-curing. However, thermoplastics often have lower overall strength than thermosets and are typically susceptible to dissolution in one or more solvents. Hydrogels are hydrophilic polymeric networks that can absorb large amounts of water without dissolving. As the hydrogel takes on water, the polymer swells as water molecules become entrained within the three-dimensional network of the polymer. The ability of hydrogels to retain large amounts of water has led to their use in a variety of applications including drug delivery, tissue engineering and wound healing. Although various medical devices may be prepared by machining, cutting, or otherwise sculpting hydrogel materials, the construction of such devices is limited because different hydrogel articles may not be directly joined together and, in some cases, hydrogels exhibit high degrees of variability in material properties.

Hydrogels have also been employed as carriers for delivery of biologically active agents. The agent is released by diffusion and/or degradation of the hydrogel. However, because the biologically active agent is typically loaded by absorption of an aqueous solution, water insoluble drugs are generally not deliverable via a hydrogel carrier. Furthermore, because the drugs are loaded into the hydrogel by a passive absorptive process, high concentrations of drug in the network are difficult to achieve. In practice however, because hydrogels generally do not possess high mechanical strength, their utility as a foundation material for biocompatible articles is limited.

Vitrimers are a class of polymers unlike either thermosets or thermoplastics. In particular, vitrimeric polymers exhibit certain properties generally associated with metals and silica glass. At elevated temperature or pressure, a vitrimer network behaves like a viscoelastic liquid. As the temperature is reduced, the topology of the network becomes fixed, and eventually the network exhibits the properties of a thermoset plastic. Like metals and glasses, vitrimer networks may be welded together using heat, which enables the production of complex objects and other articles. Similarly, fractured vitrimer networks may be rejoined using heat. However, such materials have limited utility in the biological and medical arts because the monomeric components may be toxic. Furthermore, the vitrimeric properties are accessed with high heat, thus necessitating specialized equipment and techniques. Additionally, the high temperatures required for the reshaping of the vitrimer network is incompatible with sensitive pharmaceutical and biological compounds that may be incorporated therein.

There is a need for materials that have the mechanical strength of epoxy resins, the biocompatibility of hydrogels and the dynamic capabilities of vitrimers. Accordingly, improved compositions and materials are needed.

### SUMMARY OF THE INVENTION

The present invention generally relates to compositions comprising polymeric materials. Certain of the compositions described herein include a therapeutic agent. Some of the compositions described herein are capable of thermal reconfiguration.

In one aspect, compositions are provided. In some embodiments, the composition comprises a crosslinked polymeric material and an active substance associated with the material, wherein the crosslinked polymeric material comprises a polymer backbone and between 1 mol % and 25 mol % with respect to polymer agent a food grade catalyst, inclusive, wherein the active substance is present in the composition in an amount of at least about 0.1 wt % based on the weight of the composition, and wherein the composition comprises less than about 10 wt % auxiliary materials other than the crosslinked polymeric material, food grade catalyst, and the active substance, based on the weight of the composition.

In certain embodiments, the composition comprises a crosslinked polymeric material formed by the reaction of one or more polyfunctional monomers and a food grade catalyst, wherein the polymeric material comprises a bioresponsive bond, and wherein the composition comprises less than about 10 wt % auxilliary materials other than the crosslinked polymeric material, the food grade catalyst, and, optionally, an active substance, based on the weight of the composition.

In some embodiments, the composition comprises a covalently crosslinked polymeric material formed by the reaction of a first polyfunctional monomer and a second polyfunctional monomer in the presence of a food grade catalyst wherein the first polyfunctional monomer comprises a first reactive group, wherein the second polyfunctional monomer comprises a second reactive group capable of forming a covalent bond with the first reactive group, and wherein at least about 1% of the first reactive groups in the crosslinked polymeric material are free.

In certain embodiments, the composition comprises a covalently crosslinked polymeric material, wherein the covalently crosslinked polymeric material comprises a bioresponsive bond and is formed via a reaction catalyzed by a food-grade catalyst, wherein the covalently crosslinked polymeric material comprises at least about 1% free reactive groups, and wherein the covalently crosslinked polymeric material is thermally reconfigurable.

Other advantages and novel features of the present invention will become apparent from the following detailed description of various non-limiting embodiments of the invention when considered in conjunction with the accompanying figures. In cases where the present specification and a document Incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-F are reaction schemes for forming polymeric materials, according to one set of embodiments;

FIG. 1G is a set of exemplary active substances, according to one set of embodiments;

FIGS. 1H and 1J are stacked FT-IR spectra (FIG. 1H) and ¹H NMR (DMSO-d6) spectra (FIG. 1J) taken during gel formation from time t=0 to 600 minutes during reaction. The FT-IR spectra show the appearance of the signature ester peak at 1755 cm⁻¹ (denoted by the symbol #) and the 1H NMR spectra show the disappearance of the carboxylic acid peak at 12.5 ppm (denoted by the symbol *), according to one set of embodiments;

FIG. 2A-2C are graphs showing absorption of solvent (percent weight gain) as a function of time for water (FIG. 2A); dimethyl sulfoxide (DMSO, FIG. 2B); and acetonitrile (ACN, FIG. 2C) for a polymeric material prepared with 5% catalyst, according to one set of embodiments;

FIG. 3A-3C are graphs showing absorption of solvent (percent weight gain) as a function of time for water (FIG. 3A); dimethyl sulfoxide (DMSO, FIG. 3B); and acetonitrile (ACN, FIG. 3C) for a polymeric material prepared with 5%, 10%, 15%, 20%, and 25% catalyst, according to one set of embodiments;

FIG. 4. is a graph showing a representative response of PEG.CA, PEG/PPO.CA, and PPO.CA to 24 hour incubation in simulated biologic solutions (SGF, SIF, PBS) and organic solvents (EtOH, EtOAc, hexanes) as measured by percent mass change, according to one set of embodiments;

FIG. 5. is a graph showing hydration kinetics of materials in simulated biologics (SGF, SIF, PBS) over time (t=0 to 5 hours) as measured by percent mass change, according to one set of embodiments;

FIG. 6 is a photograph of articles that can be manufactured with a polymeric material that has been impregnated with a drug, according to one set of embodiments;

FIG. 7A-C are photographs showing the method of manipulating the polymeric material, according to one set of embodiments;

FIG. 8 is a photograph showing the manipulation of the polymeric material over a period of twenty hours, according to one set of embodiments;

FIG. 9 is a photograph and magnified image showing the welding of a fractured article of the polymeric material, according to one set of embodiments;

FIG. 10A shows a scanning electron microscopy image of an exemplary textured surface, according to one set of embodiments;

FIG. 10B shows static contact angles of the surface of silicon molded PEG, PEG-PPO, and PPO polymeric materials having no specific texturing (top) and having lotus texturing (middle) on the surface. SEM images (bottom) show the surface of the lotus leaf textured polymeric materials, according to one set of embodiments;

FIG. 11 shows a plot of a tensile stress versus strain for PPO, PEG-PPO, and PPO polymeric materials, according to one set of embodiments;

FIG. 12 shows a plot of mean tensile elastic modulus as a function of strain for PPO, PEG-PPO, and PPO polymeric materials, according to one set of embodiments;

FIG. 13 shows a plot of a compressive stress versus strain for PPO, PEG-PPO, and PPO polymeric materials, according to one set of embodiments;

FIG. 14 shows a plot of mean compressive elastic modulus as a function of strain for PPO, PEG-PPO, and PPO polymeric materials, according to one set of embodiments;

FIG. 15 shows a plot of shear stress vs. shear rate for PPO, PEG-PPO, and PPO polymeric materials, according to one set of embodiments;

FIGS. 16A-16H show cyctoxicity data for HeLa (FIG. 16A), HEK293 (FIG. 16B), HT29-MTX-E12 (FIG. 16C), and C2BBe1 (FIG. 16D) cells grown on PEG polymeric materials and, for HeLa (FIG. 16E), HEK293 (FIG. 16F), HT29-MTX-E12 (FIG. 16G), and C2BBe1 (FIG. 16H) cells grown on PPO polymeric materials, for PPO, PEG-PPO, and PPO polymeric materials, according to one set of embodiments;

FIG. 17 shows a microscope photograph of HeLa cells grown on a PPO:CA:delta-decalactone polymeric material, according to one set of embodiments;

FIG. 18A shows an HPLC plot of artesunate released from a PEG:CA polymeric material, according to one set of embodiments;

FIG. 18B is a plot of cumulative mass (mg) vs. time (days) for the release of ivermectin from a PEG:CA polymeric material, according to one set of embodiments;

FIG. 18C is a plot of concentration vs. time (hours) for the release of dexamethasone in various polymeric materials, according to one set of embodiments; and

FIG. 19 is a plot of force of detachment for various polyfunctional monomers incorporated into a polymeric material, according to one set of embodiments.

### DETAILED DESCRIPTION OF THE INVENTION

Compositions and devices for bio-related and other applications have been developed. In one aspect, the compositions and devices comprise a releasable therapeutic agent. In some cases, the compositions have advantageous combinations of properties including mechanical strength, biocompatibility, moldability, and/or thermal reconfigurability. In one embodiment, the composition comprises a polymeric material and therapeutic agent associated with the polymeric material. It has been discovered that this composition advantageously can provide controlled release of the therapeutic agent, while comprising little to no auxiliary materials (e.g., solvents, catalysts, excipients) which, in some cases, may be toxic agents. In some embodiments, the composition is formed by the reaction of one or more monomers in the presence of a food grade catalyst. The use of food grade catalysts to form polymeric materials offers several advantages including, for example, the formation of compositions which contain primarily (or only) FDA approved ingredients and biocompatibility. In certain embodiments, the polymeric material comprises ester bonds such that, for example, the polymeric material is biodegradable. In another embodiment, the composition comprises a polymeric material capable of undergoing thermal reconfiguration (i.e. a dynamic network). Advantageously, the compositions and materials described herein may comprise a reconfigurable polymeric material (e.g., a thermoset polymeric material) having the strength and integrity of epoxy resins, the biomedical applicability of hydrogels, and/or the moldability of vitrimers.

The polymeric materials described herein may offer several advantages over traditional polymeric materials (e.g., for drug delivery and/or biological applications) including formation at relatively low temperatures (e.g., at or about room temperature, or less than about 90° C.) as compared to other such polymeric materials such as vitrimers, containing only materials that are FDA approved, high therapeutic agent loading (i.e. high concentrations of therapeutic agents) (e.g., such that the therapeutic agent is protected until desired time of delivery), controllable release kinetics, and/or containing materials that do not have adverse physiological effects (e.g., do not have horomone-like properties). Additionally, the compositions and polymeric materials described herein are generally formed in the presence of mild base catalysis, under relatively mild conditions (e.g., relatively low temperature), using transesterification reactions, and/or do not require post processing.

The compositions and materials described herein may be useful for a variety of applications, including drug delivery, biological diagnostics, medical devices, tissue engineering, veterinary applications, food packaging and environmental engineering applications, as described in more detail below.

Compositions are described herein. In some embodiments, the composition comprises a polymeric material. In some embodiments, the polymeric material is cross-linked. In certain embodiments, the polymeric material is amorphous. In one embodiment, the polymeric material is a derived from oligomeric or polymeric strands or chains which have undergone crosslinking. The polymeric material may be softer than conventional hardened resins and may be characterized by a lower Young's modulus and crosslinking density than conventional hardened resins. In contrast to a shape memory polymer which generally returns to its original form after it has been stretched or otherwise stressed, the polymeric material disclosed herein remains fixed in its new shape after it has been molded into a new position.

In one embodiment, the polymeric material is thermally reconfigurable. For example, the polymeric material, in some cases, undergoes an observable dynamic equilibrium (i.e. thermal configuration) at elevated temperatures. In some embodiments, the polymeric material may have a particular shape, and is mechanically deformed to form a new shape, such that upon heating to an elevated temperature (e.g., greater than about 40° C., greater than about 60° C., greater than about 90° C.), the polymeric material maintains the new shape (e.g., at the elevated temperature and/or when cooled). In some embodiments, the polymeric material is a thermally reconfigurable thermoset polymer. Mechanically deforming to form a new shape includes, for example, bending, twisting, folding, molding (e.g., pressing the material into a mold having a new shape), expanding (e.g., applying a tensile force to the material), compressing, and/or wrinkling the polymeric material. In some embodiments, the polymeric material may be broken (e.g., torn, cut) into two or more pieces and returned to one piece via thermal reconfiguration. For example, in some such embodiments, the polymeric material may be broken (e.g., forming at least two pieces of the original polymeric material), one or more of the broken surfaces may be rejoined and heat applied to the rejoined surfaces such that the polymeric material reforms into a single piece.

The thermal reconfiguration (e.g., dynamic equilibrium) generally involves the formation and destruction of covalent bonds. In certain embodiments the thermal reconfiguration processes does not substantially change the overall number of bonds in the polymeric material. In these embodiments, the mechanical strength of the polymeric material is approximately the same before and after undergoing the thermal reconfiguration. In other embodiments the thermal reconfiguration process results in a different number of bonds in the material after undergoing the thermal reconfiguration than before. In some such embodiments, the mechanical strength of the polymeric material may be changed after undergoing a thermal reconfiguration. In some cases, thermal reconfiguration may occur above a particular temperature (e.g., greater than about 40° C., greater than about 60° C., greater than about 90° C.). In certain embodiments, the particular temperature at which thermal reconfiguration may occur is sufficient to induce transesterification.

In some embodiments, the polymeric material is cross-linked. In certain embodiments, the polymeric material is covalently cross-linked. In some embodiments, the polymeric material is formed by the reaction of two or more polyfunctional monomers (e.g., a first polyfunctional monomer and a second polyfunctional monomer). In certain embodiments, the polymeric material is formed by the reaction of two or more, three or more, four or more, or five or more polyfunctional monomers. In some embodiments, each polyfunctional monomer comprises a reactive functional group. In certain embodiments, two or more reactive functional groups may form a covalent bond with one another. For example, in some cases, the reaction of a first reactive functional group and a second reactive functional group forms a covalent bond between the first reactive functional group and the second reactive functional group. In other embodiments, the reaction between two or more reactive functional groups is a Michael-addition. In other embodiments, the reaction between two or more reactive functional groups is a cycloaddition reaction, especially a Diels-Alder reaction.

In some embodiments, one or more polyfunctional monomers is bifunctional. In certain embodiments, one or more polyfunctional monomers is trifunctional. In some cases, one or more polyfunctional monomers may be tetrafunctional, pentafunctional, hexafunctional, or have higher orders of functionality. In a particular embodiments, the polymeric material is formed by the reaction of one or more bifunctional monomers and one or more trifunctional monomers.

In one embodiment, the polymeric material (e.g., the covalently crosslinked polymeric material) may be represented by Formula (I).

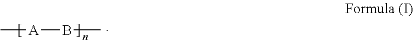

wherein A is derived from at least one polyfunctional monomer containing at least two reactive functional groups, and B is derived from at least one polyfunctional monomer containing at least two reactive functional groups, and wherein the compound of Formula (I) comprises crosslinked bonds. For example, in a particular embodiment, the polymeric material comprising the structure as in Formula (I) is formed by the reaction of a first polyfunctional monomer comprising two reactive functional groups and a second polyfunctional comprising three reactive functional groups. In another embodiment, the polymeric material comprising the structure as in Formula (I) is formed by the reaction of a first polyfunctional monomer comprising two reactive functional groups, a second polyfunctional monomer different than the first polyfunctional monomer comprising two reactive functional groups, and a third polyfunctional monomer comprising three reactive functional groups. In some such embodiments, the reactive functional groups of the first polyfunctional monomer may be the same or different as the reactive functional groups of the second polyfunctional monomer and/or the third polyfunctional monomer. For example, the reactive groups of the first polyfunctional monomer may react with (and form a covalent bond with) the reactive groups of the second polyfunctional monomer and/or the third polyfunctional monomer.

In some embodiments, one or more polyfunctional monomers contain an oligomeric moiety. In certain embodiments, the polymeric material of Formula (I) is further characterized by the presence of at least two reactive groups capable of forming a crosslink bond.

In some embodiments, one or more reactive functional groups will participate in a thermal reconfiguration (e.g., dynamic equilibrium) chemical reaction with each other at the temperatures specified above. In certain embodiments, the reaction of two or more polyfunctional monomers results in the formation of at least one reactive functional group capable of participating in a crosslink. In some embodiments, one or more polyfunctional monomers contain reactive functional groups capable of forming a crosslink.

In some embodiments, the polymeric material comprises at least two different types of reactive functional groups. When the polymeric material is supplied with heat or pressure, some of these functional groups undergo chemical bond formation forming new links in the polymeric material, while other chemical bonds in the polymeric material are broken, resulting in the formation of reactive functional groups. The particular characteristics of the polymeric material may be due in part, for example, to thermal reconfiguration (or dynamic equilibrium) processes within the polymeric material.

In certain embodiments, the thermal reconfiguration process is a nucleophilic substitution reaction. In some cases, the thermal reconfiguration process may be a transesterification reaction. In other embodiments, the polymeric material is capable of undergoing two or more orthogonal thermal reconfiguration processes. In certain embodiments, the polymeric material backbone contains one type of reactive functional group capable of undergoing a thermal reconfiguration processes, and one or more pendent groups contain a different type of reactive functional group capable of undergoing a different thermal reconfiguration process.

In certain embodiments, the compound of Formula (I) is prepared by combining two or more polyfunctional monomers, and then incubating the mixture at a temperature sufficient to initiate polymerization to reach the gel point. In some embodiments, the two or more polyfunctional monomers are combined in the presence of a catalyst. In certain embodiments, two or more polyfunctional monomers are combined in the presence of a subunit compound, in the presence of an active substance, or both.

In some embodiments, the polyfunctional monomer has a structure as in Formula (II):

Q¹-L-Q² (II)

wherein Q¹ and Q² are the same or different and a reactive functional group and L has a structure as in Formula (III):

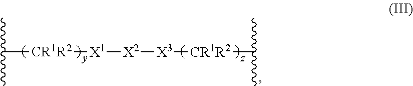

wherein [symbol] indicates a point of connection to Q¹ and Q².

In some embodiments, the polyfunctional monomer has a structure as in:

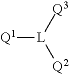

wherein Q¹, Q², and Q³ are the same or different and a reactive functional group and L has a structure as in Formula (III).
In some embodiments, X¹, X², and X³ are the same or different and are absent or selected from the group consisting of (CR¹R²)ₘ, a heteroatom, an alkenyl, an alkynyl, a cycloalkyl, an aryl, a heterocyclic group, a heteroaryl group, and an oligomeric group. In certain embodiments, X¹, X², and/or X³ are absent.

In certain embodiments, m is zero or any integer. For example, in some embodiments, m is 0. In certain embodiments, m is 1-3, 2-4, 3-6, 4-8, 5-10, 8-16, 12-24, 20-30, 25-50, 40-60, 50-100, 75-150, 125-200, 150-300, 250-500, 400-600, 500-800, or 750-1500. In some cases, m is 1-3. In certain embodiments, m is 2-4. In some cases, m is 4-8. In some embodiments, m is 8-16. The value of m may be selected to impart certain properties in the polymeric material (e.g., crosslink density, Young's elastic modulus).

In some embodiments, y is zero or any integer. For example, in some embodiments, y is 0. In certain embodiments, y is 1-3, 2-4, 3-6, 4-8, 5-10, 8-16, 12-24, 20-30, 25-50, 40-60, 50-100, 75-150, 125-200, 150-300, 250-500, 400-600, 500-800, or 750-1500. In some cases, y is 1-3. In certain embodiments, y is 2-4. In some cases, y is 4-8. In some embodiments, y is 8-16. The value of y may be selected to impart certain properties in the polymeric material (e.g., crosslink density, Young's elastic modulus).

In certain embodiments, z is zero or any integer. For example, in some embodiments, z is 0. In certain embodiments, z is 1-3, 2-4, 3-6, 4-8, 5-10, 8-16, 12-24, 20-30, 25-50, 40-60, 50-100, 75-150, 125-200, 150-300, 250-500, 400-600, 500-800, or 750-1500. In some cases, z is 1-3. In certain embodiments, z is 2-4. In some cases, z is 4-8. In some embodiments, z is 8-16. The value of z may be selected to impart certain properties in the polymeric material (e.g., crosslink density, Young's elastic modulus).

In a particular embodiment, m+y+z is zero. In certain embodiments, m+y+z is 1. In some cases, m+y+z is an integer and is 2 or greater.

In some embodiments, each R¹ and R² are the same or different and are selected from the group consisting of hydrogen, an aliphatic group, a halogen, a hydroxyl, a carbonyl, a thiocarbonyl, an oxo, an alkoxy, an epoxy, a phosphoryl, a phosphate, a phosphonate, a phosphinate, an amino, an amido, an amidine, an imine, a cyano, a nitro, an azido, a thiol, an alkylthio, a sulfate, a sulfonate, a sulfamoyl, a sulfonamido, a sulfonyl, a cycloalkyl, a heterocyclyl, an aralkyl, and an aromatic or heteroaromatic or a Michael acceptor, wherein any two or more R¹ and R² groups may be bonded together so as to form a ring system. In certain embodiments, each R¹ and/or R² may be Q³ (i.e. a reactive functional group).

In an exemplary embodiment, the polyfunctional monomer has the structure as in Formula (IV):

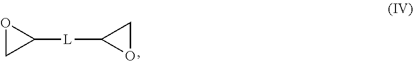

wherein L is as described above. In another exemplary embodiments, the polyfunctional monomer has the structure as in:

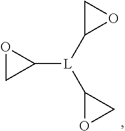

wherein L is as described above. In yet another exemplary embodiment, the polyfunctional monomer has a structure as in Formula (V) or Formula (VI):

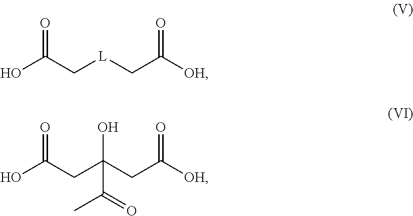

wherein L is described above. In some embodiments, the polymeric material is formed by the reaction of a first polyfunctional monomer having a structure as in Formula (IV) with a second polyfunctional monomer having a structure as in Formula (V) or Formula (VI).

Polyfunctional monomers described herein may comprise at least two, at least three, at least four, or at least five reactive functional groups. For example, in some embodiments, Q¹, Q², and Q³ may be the same or different and an electrophilic functional groups or a nucleophilic functional group.

In some embodiments, one or more reactive groups (e.g., Q¹, Q², and/or Q³) is an electrophilic functional groups. For example, a monomer may comprise at least two, at least three, at least four, or at least five electrophilic functional groups. Non-limiting examples of suitable electrophilic functional groups include alkenes, alkynes, esters (e.g., N-hydroxysuccinimide ester), acrylates, methacrylates, acyl halides, acyl nitriles, alkyl halides, aldehydes, ketones, alkyl sulfonates, anhydrides, epoxides, haloacetamides, aziridines, and diazoalkanes.

In certain embodiments, one or more reactive functional groups (e.g., Q¹, Q², and/or Q³) is a nucleophilic functional groups. For example, a monomer may comprise at least two, at least three, at least four, or at least five nucleophile reactive functional groups. Non-limiting examples of suitable nucleophilic functional groups include alcohols, amines, anilines, phenols, hydrazines, hydoxylamines, carboxylic acids, alkoxide salts, alkenes, thiols, and glycols.

The polyfunctional monomers described herein may comprise at least one electrophilic functional group and at least one nucleophilic functional group. For example, in an exemplary embodiment, the first polyfunctional monomer comprises both an electrophilic functional group and a nucleophilic functional group. In certain embodiments, the first polyfunctional monomer comprises two or more electrophile functional groups and the second polyfunctional monomer comprises two or more nucleophile functional groups.

In some cases, the reaction of an electrophilic functional group and a nucleophilic functional group form a bioresponsive bond such as an ester bond, an ether bond, an amide bond, an amine bond, or a thioether bond. For example, in certain embodiments, the polymeric material comprises an ester bond formed by the reaction of an electrophilic functional group and a nucleophilic functional group. In some embodiments, the polymeric material comprises an ether bond formed by the reaction of an electrophilic functional group and a nucleophilic functional group. Other bonds are also possible.

In an exemplary embodiment, the first polyfunctional monomer is selected from a compound of Formula (VII):

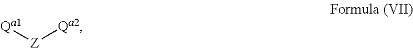

wherein Q^{a1} and Q^{a2} are electrophilic functional groups and Z has a structure selected from:

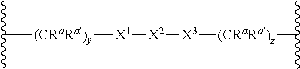

wherein [symbol] indicates a point of connection to Q^{a1} and Q^{a2} to Z,
wherein y and z are in each case independently selected from zero or any integer, as described above,
wherein R^{a} and R^{a′} are in each case independently selected from hydrogen, Q^{a3} (e.g., an electrophilic functional group or a nucleophilic functional group), an aliphatic group, a halogen, a hydroxyl, a carbonyl, a thiocarbonyl, an oxo, an alkoxy, an epoxy, a phosphoryl, a phosphate, a phosphonate, a phosphinate, an amino, an amido, an amidine, an imine, a cyano, a nitro, an azido, a thiol, an alkylthio, a sulfate, a sulfonate, a sulfamoyl, a sulfonamido, a sulfonyl, a cycloalkyl, a heterocyclyl, an aralkyl, or an aromatic or heteroaromatic or a Michael acceptor, wherein any two or more R^{a} and R^{a′} groups may be bonded together so as to form a ring system;
wherein X¹, X² and X³ are independently selected from
1) (CR^{a}R^{a′})ₘ, wherein m is selected from 1-20,
2) a heteroatom,
3) an alkenyl group of the formula:

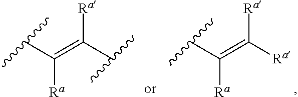

4) an alkynyl group of the formula:

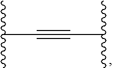 [SMILES: *C#C*]

5) a cycloalkyl group,
6) an aryl group,
7) a heterocyclic group,
8) a heteroaryl group,
9) an oligomeric group, and
wherein any of X¹, X² and X³ may also be absent. The cycloalkyl, aryl, heterocyclyl and heteroaryl groups may each be substituted or unsubstituted. In some embodiments, if X¹, X² and X³ are all absent, either y or z is not zero. In certain embodiments, if y and z are each zero, then at least one X¹, X² or X³ is present.

In some embodiments, z and y are selected from 0-20 (e.g., 0-10).

In certain embodiments, the first polyfunctional monomer is selected from a single compound of Formula (VII). In other embodiments, the first polyfunctional monomer and a third polyfunctional monomer is a mixture of two or more different compounds of Formula (VII). For embodiments in which the first polyfunctional monomer and the third polyfunctional monomer is a mixture of two or more compounds, the first polyfunctional monomer and the third polyfunctional monomer may be a mixture of two or three different compounds of Formula (VII).

In certain embodiments, Q^{a1}, Q^{a2}, and/or Q^{a3} are independently selected from:

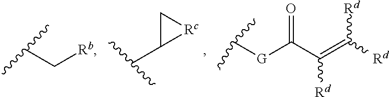

wherein R^{b} is a leaving group, R^{c} is a heteroatom or NR^{a1}, G is a heteroatom or aliphatic group, and R^{d} is independently selected from hydrogen and aliphatic. In certain embodiments, R^{b} is a halogen, sulfonyloxyaryl or sulfonyloxyalkyl leaving group, R^{c} is oxygen, nitrogen, or sulfur, G is oxygen or NH, and R^{d} is independently hydrogen or methyl. In some embodiments, Q^{a1}, Q^{a2}, and/or Q^{a3} are independently selected from diglycidyl, acrylate, methacrylate, acrylamide and methacrylamide:

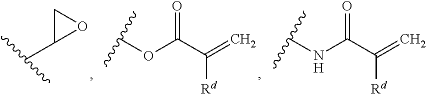

R^{d}=hydrogen or methyl

In a particular embodiment, Q^{a1} and Q^{a2} are the same. In certain embodiments, Q^{a1}, Q^{a2}, and/or Q^{a3} are the same. In some cases, Q^{a1}, Q^{a2}, and/or Q^{a3} are different.

In certain embodiments, X¹ and X³ are absent and X² is selected from an oligomeric monomer comprising an oligomer and the structure as in Formula (V):

Q^{a1}-(CR^{a}R^{a′})_{y}-oligomer-(CR^{a}R^{a′})_{z}-Q^{a2} (V)

Non-limiting examples of suitable oligomers include naturally occurring polysaccharides, non-naturally occurring polysaccharide, polyacrylates, polymethacrylates, polyvinyl alcohols, polyalkylene glycols, polyacrylamides, polyvinylpyrrolidones, polyurethanes, polylactides, lactide/glycolide copolymers, polycaprolactones, polydioxanones, polyanhydrides, polyhydroxybutyrates, polysiloxanes, and polytrimethylene carbonates.

In some embodiments, the oligomer is a polyalkylene oxide such as polyalkylene glycol. In one embodiment, the oligomer is polyethylene glycol. In another embodiment, the oligomer is polypropylene glycol. In some embodiments, the polyethylene glycol or a polypropylene glycol has an average molecular weight between 2-50 Daltons, 20-300 Daltons, 200-1000 Daltons or between 300-700 Daltons.

In certain embodiments, the first polyfunctional monomer has a structure as in Formula (VIII):

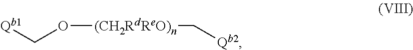

wherein each R^{d} and R^{e} are the same or different and selected from hydrogen, methyl, alcohol, or carboxylic acid. In some cases, n is selected to give an average molecular weight between 2-50 Daltons, 20-300 Daltons, 200-1000 Daltons or between 300-700 Daltons. In certain embodiments, n is 1-3, 2-4, 3-6, 4-8, 5-10, 8-16, 12-24, 20-30, 25-50, 40-60, 50-100, 75-150, 125-200, 150-300, 250-500, 400-600, 500-800, or 750-1500. In some cases, n is 1-3. In certain embodiments, n is 2-4. In some cases, n is 4-8. In some embodiments, n is 8-16. The value of n may be selected to impart certain properties in the polymeric material (e.g., crosslink density, Young's elastic modulus).
In some embodiments, the first polyfunctional monomer has a structure as in Formula (IX):

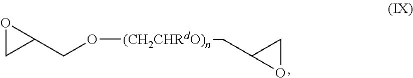

and is a diglycidyl monomer.

In some such embodiments, when R^{d} is hydrogen, the first polyfunctional monomer may be designated diglycidyl PEG, and when R^{d} is methyl, the first polyfunctional monomer may be designated diglycidyl PPO.

In some embodiments, the oligomer may be a branched oligomer. In certain embodiments, the branched oligomer is a four-arm or eight-arm polyalkylene glycol. In some embodiments, the first polyfunctional monomer is a tetraglycidyl polyethylene glycol or a tetraglycidyl polypropylene glycol.

In another embodiment, first polyfunctional monomer may comprise the structure as in Formula (X):

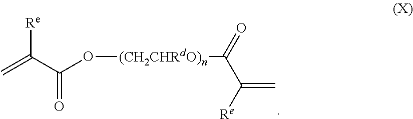

In some such embodiments, when R^{d} and R^{e} are both hydrogen, the first polyfunctional monomer may be designated PEG diacrylate, when R^{d} is hydrogen and R^{e} are methyl, the first polyfunctional monomer may be designated PEG dimethacrylate, when R^{d} and R^{e} are both methyl, the first polyfunctional monomer may be designated PPO dimethacrylate and when R^{d} is methyl and R^{e} is hydrogen, the first polyfunctional monomer may be designated PPO diacrylate.

In another embodiment, the first polyfunctional monomer may comprise the structure as in Formula (XI):

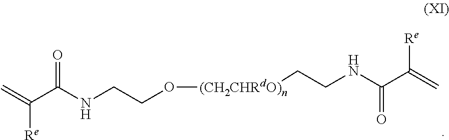

In some such embodiments, when R^{d} and R^{e} are both hydrogen, the first polyfunctional monomer may be designated PEG diacrylamide, when R^{d} is hydrogen and R^{e} are methyl, the first polyfunctional monomer may be designated PEG dimethacrylamide, when R^{d} and R^{e} are both methyl, the first polyfunctional monomer may be designated PPO dimethacrylamide and when R^{d} is methyl and R^{e} is hydrogen, the first polyfunctional monomer may be designated PPO diacrylamide. Further embodiments of the first polyfunctional monomer include four-arm and eight-arm polyalkylene oligomers containing a Michael acceptor. In some cases, R^{d} is an electrophilic functional group. In certain embodiments, R^{d} is a nucleophilic functional group.

In some embodiments, the first polyfunctional monomer comprises a polyalkylene moiety and has an average molecular weight of 100-10,000 Daltons. In another embodiment, the first polyfunctional monomer comprises a polyalkylene moiety and has an average molecular weight of 50-200 Daltons, 100-5,000 Daltons, or 100-1,000 Daltons, or 300-700 Daltons.

In some embodiments, (CR^{a}R^{a′})_{y} and (CR^{a}R^{a′})_{z} are selected so as to be methylene—(CH₂)—:

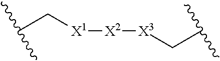

wherein [symbol] indicates a point of connection to Q^{a1} and Q^{a2},
wherein X¹, X² and X³ are independently selected from
1) (CR^{a}R^{a′})ₘ,
2) a heteroatom,
3) an alkenyl group of the formula:

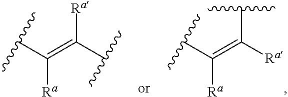

4) an alkynyl group of the formula:

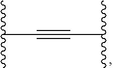 [SMILES: *C#C*]

5) a cyclo alkyl group,
6) an aryl group,
7) a heterocyclic group, and
8) a heteroaryl group,
wherein R^{a} and R^{a′} are in each case independently selected from hydrogen, Q^{a3} (e.g., an electrophilic functional group or a nucleophilic functional group), an aliphatic group, a halogen, a hydroxyl, a carbonyl, a thiocarbonyl, an oxo, an alkoxy, an epoxy, a phosphoryl, a phosphate, a phosphonate, a phosphinate, an amino, an amido, an amidine, an imine, a cyano, a nitro, an azido, a thiol, an alkylthio, a sulfate, a sulfonate, a sulfamoyl, a sulfonamido, a sulfonyl, a cycloalkyl, a heterocyclyl, an aralkyl, or an aromatic or heteroaromatic or a Michael acceptor, wherein any two or more R^{a} and R^{a′} groups may be bonded together so as to form a ring system.

In certain embodiments, X¹ and X³ are selected from heteroatom, and X² is (CR^{a}R^{a′})ₘ, wherein m is an integer greater than zero and R^{a} and R^{a′} are as defined above. In a particular embodiment, the first polyfunctional monomer is trimethyolpropane triglycidyl ether:

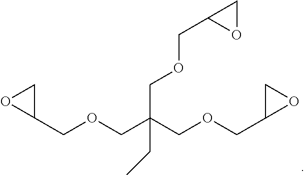 [SMILES: CCC(COCC1CO1)(COCC1CO1)COCC1CO1]

Other non-limiting examples of suitable first polyfunctional monomers include 1,4 butanediol diglycidyl ether, neopentyl diglycidyl ether, alkylene diacrylamide, alkylene diacrylate (e.g., comprising an alkylene group having from 1-6 carbon atoms or 1-3 carbon atoms) alkylene dimethacrylamide and alkylene dimethacrylate. For example, in some embodiments the first polyfunctional monomer is methylene bisacrylamide, 1,2 ethylenebisacrylamide, 1,1 ethyldiacrylamide, 1,3 propylenebisacrylamide, as well as the corresponding methacrylamides, acrylates and methacrylates.

In another embodiment, Z is selected from:

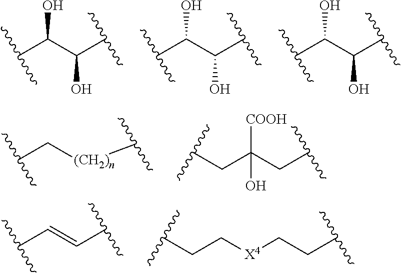

wherein X⁴ is oxygen, nitrogen, sulfur or disulfide (—S—S—) and wherein n is 0-10 (e.g., 0, 1, 3, or). In certain embodiments, X⁴ is oxygen.

Many of the above embodiments of the first polyfunctional monomers are commercially available from suppliers such as Sigma-Aldrich, Acros, and Creative PEGWorks. Polyfunctional monomers which are not commercially available may be obtained using synthetic protocols known to those of skill in the art. For instance, many diglycidyl compounds may be obtained by reacting the corresponding dihydroxy compound with epichlorohydrin and/or glycerol. Similarly, many diacrylate and diacrylamide compound can be prepared from the dihydroxy or diamine compound with an activated acrylic or methacrylic acid derivative. Other epoxides may be obtained by epoxidation of the corresponding olefin.

In certain embodiments, the first polyfunctional monomer does not contain an amino (e.g., NH or NH₂) functional group. In some embodiments, the second polyfunctional monomer does not contain an amino (e.g., NH or NH₂) functional group.

In one embodiment, the second polyfunctional monomer comprises the structure as in Formula (XII):

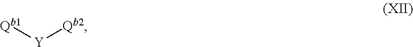

wherein O^{b1} and Q^{b2} are nucleophilic functional groups and Y has a structure selected from:

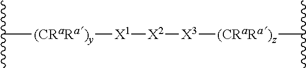

wherein [symbol] indicates a point of connection to O^{b1} and Q^{b2} to Y,
wherein y and z are in each case independently selected from zero or any integer as described above,
wherein each R^{a} and R^{a′} are in each case independently selected from hydrogen, Q^{b3} (e.g., an electrophilic functional group or a nucleophilic functional group), an aliphatic group, a halogen, a hydroxyl, a carbonyl, a thiocarbonyl, an oxo, an alkoxy, an epoxy, a phosphoryl, a phosphate, a phosphonate, a phosphinate, an amino, an amido, an amidine, an imine, a cyano, a nitro, an azido, a thiol, an alkylthio, a sulfate, a sulfonate, a sulfamoyl, a sulfonamido, a sulfonyl, a cycloalkyl, a heterocyclyl, an aralkyl, or an aromatic or heteroaromatic or a Michael acceptor, wherein any two or more R^{a} and R^{a′} groups may be bonded together so as to form a ring system;
wherein X¹, X² and X³ are independently selected from
1) (CR^{a}R^{a′})ₘ, wherein m is selected from 1-20,
2) a heteroatom,
3) an alkenyl group of the formula:

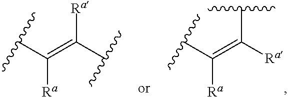

4) an alkynyl group of the formula:

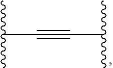 [SMILES: *C#C*]

5) a cycloalkyl group,
6) an aryl group,
7) a heterocyclic group,
8) a heteroaryl group,
9) an oligomeric group, and
wherein any of X¹, X² and X³ may also be absent. The cycloalkyl, aryl, heterocyclyl and heteroaryl groups may each be substituted or unsubstituted. In some embodiments, if X¹, X² and X³ are all absent, either y or z is not zero. In certain embodiments, if y and z are each zero, then at least one X¹, X² or X³ is present.

In some embodiments, z and y are selected from 0-20 (e.g., 0-10).

In certain embodiments, the second polyfunctional monomer is selected from a single compound of Formula (XII). In other embodiments, the second polyfunctional monomer and the third polyfunctional monomer is a mixture of two or more different compounds of Formula (XII). For embodiments in which the second polyfunctional monomer and the third polyfunctional monomer is a mixture of two or more compounds, the second polyfunctional monomer and the third polyfunctional monomer may be a mixture of two or three different compounds of Formula (XII).

In certain embodiments, Q^{b1}, Q^{b2}, and/or Q^{b3} are independently selected from:

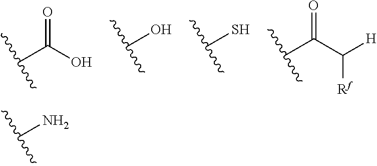

wherein R^{f} is selected from the group consisting of alkyl, alkenyl, alkynyl, cycloalkyl, heterocyclyl, aryl, aralkyl, heteroaryl, alkoxy, keto, alkyl carboxylate, and alkyl carboxylamide.

In some embodiments, Q^{b1}, Q^{b2} and/or Q^{b3} are independently selected from carboxylic acid, hydroxyl and thiol.

In certain embodiments, Q^{b1} and Q^{b2} are the same. In some embodiments, Q^{b1}, Q^{b2}, and/or Q^{b3} are the same. In certain embodiments, Q^{b1}, Q^{b2}, and/or Q^{b3} are different.

In certain embodiments, X¹ and X³ are absent and X² is selected from oligomer:

Q^{b1}-(CR^{a}R^{a′})_{y}-oligomer-(CR^{a}R^{a′})_{z}-Q^{b2}

Non-limiting examples of suitable oligomers include naturally occurring polysaccharides, non-naturally occurring polysaccharide, polyacrylates, polymethacrylates, polyvinyl alcohols, polyalkylene glycols, polyacrylamides, polyvinylpyrrolidones, polyurethanes, polylactides, lactide/glycolide copolymers, polycaprolactones, polydioxanones, polyanhydrides, polyhydroxybutyrates, and polytrimethylene carbonates.

In some embodiments, the oligomer is a polyalkylene oxide such as polyalkylene glycol. In one embodiment, the oligomer is polyethylene glycol. In another embodiment, the oligomer is polypropylene glycol. In some embodiments, the polyethylene glycol or a polypropylene glycol has an average molecular weight between 50-300 Daltons, 200-1000 Daltons, or between 300-700 Daltons.

In certain embodiments, the second polyfunctional monomer comprises a structure as in Formula (XIII):

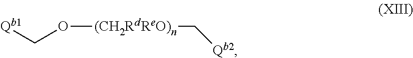

wherein R^{d} and R^{e} are the same or different and selected from hydrogen, methyl, alcohol, or carboxylic acid. In some cases, n is selected to give an average molecular weight between 50-300 Daltons, 200-1000 Daltons or between 300-700 Daltons. Other values for n are also possible, as described above.

In some embodiments, the second polyfunctional monomer contains two carboxylic acid groups separated by a linker. In some cases, the linker may be an oligomer. In certain embodiments, the second polyfunctional monomer is a diacetic acid polyalkylene glycol having the following structure:

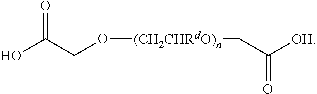

In some such embodiments, when R^{d} is hydrogen, the second polyfunctional monomer may be designated diacetic acid PEG, and when R^{d} is methyl, the second polyfunctional monomer may be designated diacetic acid PPO.

In certain embodiments, the second polyfunctional monomer comprising the structure as in Formula (XII) contains at least one R^{a} group that is a carboxylic acid.

In some embodiments, the second polyfunctional monomer comprises the structure as in Formula (XII) and Y is a linker having one of the following structures:

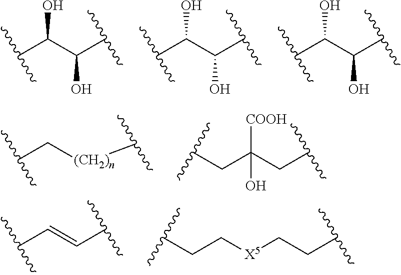

wherein X⁵ is oxygen, nitrogen, sulfur or disulfide (—S—S—) and wherein n is 0-10 (e.g., 0, 1, 3, or). In certain embodiments, X⁵ is oxygen.

In some embodiments, the second polyfunctional monomer is selected from the group consisting of maleic anhydride, succinic anhydride, tartaric acid, malonic acid, fumaric acid, succinic acid, malic acid, tartaric acid, glutaric acid, hydroxyglutaric acid, pimelic acid, sebacic acid, thiodipropionic acid, adipic acid, delta-decalactone, gamma-decalactone, caprolactone, dithiodipropionic acid, mercaptosuccinic acid, mercapto glutaric acid, and amino acids such as aspartic acid and glutamic acid. In certain embodiments, the second polyfunctional monomer is an amino acid like aspartic acid or glutamic acid, the nitrogen atom is deactivated so that it does not participate in the polymerization and/or thermal reconfiguration process. Those skilled in the art would be capable of selecting suitable deactivating groups and may include, for example, amides and carbamates, including acetamide, benzamide, tertbutyloxycarbonyl (tBOC), benzyloxycarbonyl (CBz). Other deactivating groups are also possible.

In certain embodiments, the second polyfunctional monomer comprises at least one R^{a} group that is carboxylic acid and is selected from the group consisting of citric acid, isocitric acid, cis-aconitic acid, trans-aconitic acid, and carballylic acid.

In some embodiments, the second polyfunctional monomer is food derived and/or food-grade. Non-limiting examples of food derived or food-grade polyfunctional monomers include citric acid, fumaric acid, tartaric acid, succinic acid, decalactones, adipic acid, pentadecalactone, and thiodipropionic acid. Other food derived and/or food-grade monomers are also possible.

In some embodiments, the molar ratio of the first polyfunctional monomer (e.g., comprising electrophilic reactive groups) and the second polyfunctional monomer (e.g., comprising nucleophilic reactive groups) ranges between about 10:1 and about 1:10. For example, in certain embodiments, the molar ratio of first polyfunctional monomer to second polyfunctional monomer is at less than about 10:1, less than about 8:1, less than about 6:1, less than about 4:1, less than about 2:1, less than about 1.5:1, less than about 1:1, less than about 1:1.5, less than about 1:2, less than about 1:4, less than about 1:6, or less than about 1:8. In some embodiments, the molar ratio of first polyfunctional monomer to second polyfunctional monomer is greater than or equal to about 1:10, greater than or equal to about 1:8, greater than or equal to about 1:6, greater than or equal to about 1:4, greater than or equal to about 1:2, greater than or equal to about 1:1.5, greater than or equal to about 1:1, greater than or equal to about 1.5:1, greater than or equal to about 2:1, greater than or equal to about 4:1, greater than or equal to about 6:1, or greater than or equal to about 8:1. Combinations of the above-referenced ranges are also possible (e.g., between about 10:1 and about 1:10, between about 1:4 and about 4:1, between about 1:2 and about 2:1). In an exemplary embodiment, Q^{a1} and Q^{a2} are epoxide functional groups and Q^{b1} and Q^{b2} are carboxylic acid functional groups. The combination of the first polyfunctional monomer and the second polyfunctional monomer provides a prepolymer comprising the polyester structure as in Formula (XIV):

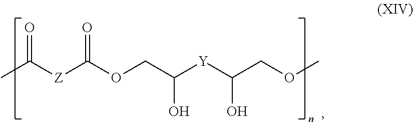

wherein n is an integer, and wherein Z and Y are defined above. In certain embodiments of the compound of Formula (XIV), at least one of Z or Y contains an oligomeric moiety. In certain embodiments of the compound of Formula (XIV), the oligomeric moiety is a polyalkylene oxide.
In the prepolymer of Formula (XIV), the alcohol is the reactive functional group resulting from the combination of the epoxide and carboxylic acid moieties. Because the prepolymer of Formula (XIV) contains two different reactive functional groups capable of reacting with each other, the prepolymer may undergo covalent crosslinking and/or associative crosslinking via hydrogen bonding (e.g., between hydroxyl groups and carboxylic acid groups). In this particular embodiment, the alcohol and ester functional groups can undergo a transesterification reaction.

As described above, in certain embodiments, the amount of the second polyfunctional monomer (e.g., having a structure as in Formula (XII)) is selected such that the number of nucleophilic groups exceeds the number of electrophilic groups in the first polyfunctional monomer (e.g., having a structure as in Formula (VII)). This embodiment may be represented by the following equation:

N_{[NUC]}>N_{[ELEC]}

In the above equation, N_{[NUC]} represents the total number of nucleophilic functional groups in the second polyfunctional monomer and N_{[ELEc]} represents the total number of electrophilic functional groups in the first polyfunctional monomer. In embodiments where the first polyfunctional monomer is a compound containing two epoxides, and the second polyfunctional monomer is a compound containing three carboxylic acids, the above relationship is satisfied when, for example, an equimolar amount of the first and second polyfunctional monomers are employed. In embodiments when the first polyfunctional monomer is a compound containing two epoxides, and the second polyfunctional monomer is a compound containing two carboxylic acids, the above relationship is satisfied when, for example, an excess amount of the second polyfunctional monomer is employed relative to the first polyfunctional monomer.

In certain embodiments, at least one of the polyfunctional monomers (e.g., the first polyfunctional monomer, the second polyfunctional monomer) contains an oligomeric moiety.

In some embodiments, the polymeric material is formed by the reaction of three or more polyfunctional monomers. For example, in some embodiments, the polymeric material is formed by the reaction of a first polyfunctional monomer and a third polyfunctional monomer each comprising a structure as in Formula (VII) with a second polyfunctional monomer comprising a structure as in Formula (XII), wherein the first polyfunctional monomer and the third polyfunctional monomer are different. In certain embodiments, the polymeric material is formed by the reaction of a first polyfunctional monomer comprising a structure as in Formula (VII) with a second polyfunctional monomer and a third polyfunctional monomer each comprising a structure as in Formula (XII), wherein the second polyfunctional monomer and the third polyfunctional monomer are different.

In some embodiments, polyfunctional monomers (or the polymeric material formed by the reaction of two or more polyfunctional monomers) are non-toxic.

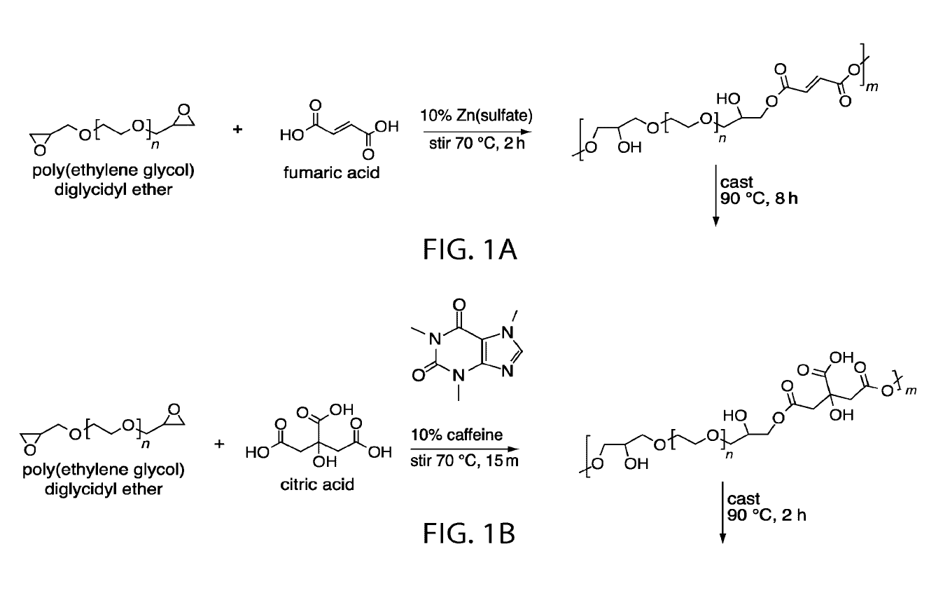

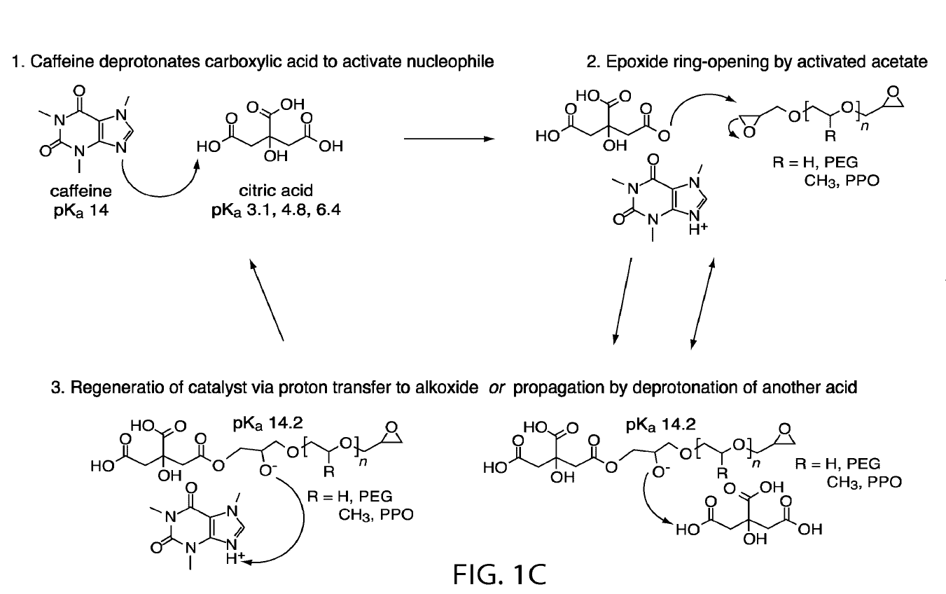

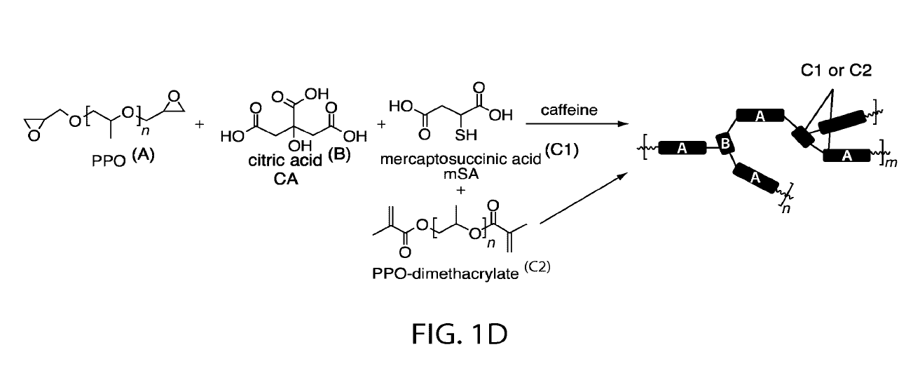

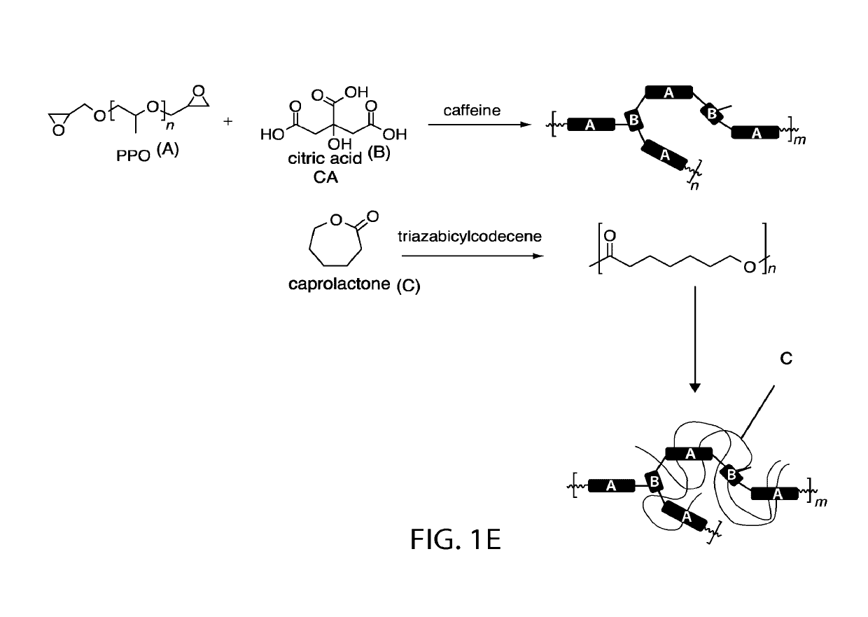

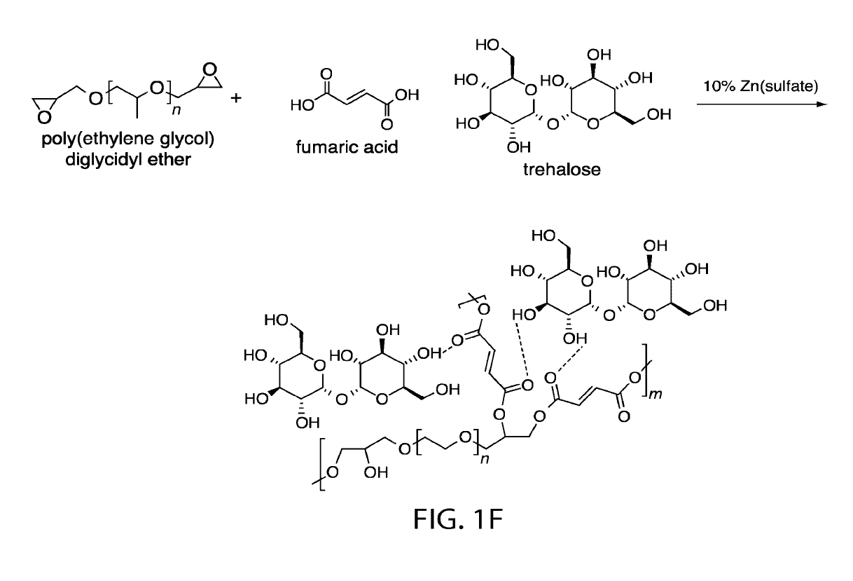

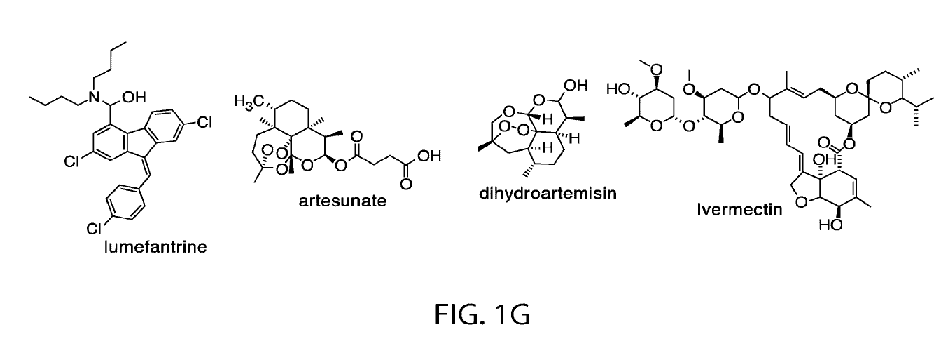

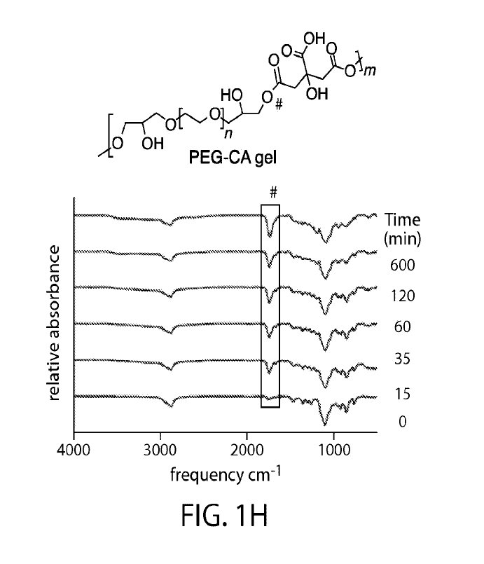

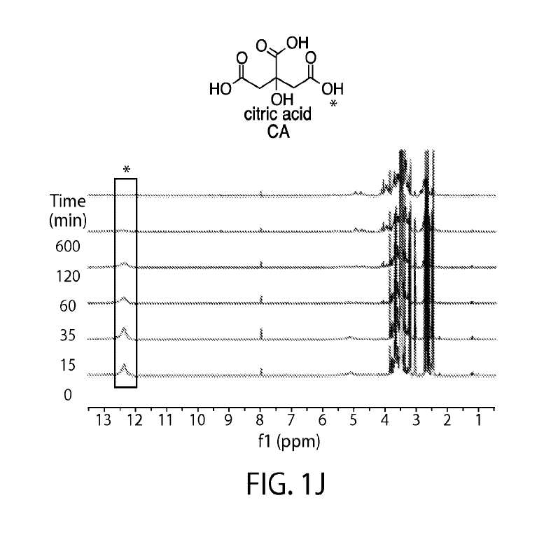

In some embodiments, a third polyfunctional monomer comprises a sugar. Non-limiting examples of suitable sugars include sucrose, trehalose, glucose, starches (e.g., tapioca, arrowroot), chitosan, alginate, guar gum, An exemplary reaction of two or more polyfunctional monomers and a sugar (e.g., trehalose) is shown in FIG. 1F.

In some embodiments, the third polyfunctional monomer comprises a structure as in Formula (II), wherein L is a particle. In certain embodiments, the additional monomeric unit comprises a particle. In some embodiments, the particle may be functionalized with one or more reactive groups. For example, the third polyfunctional monomer and/or additional monomeric unit may comprise a particle functionalized with a plurality of reactive groups. In certain cases, the particle is functionalized with a plurality of carboxylic acid reactive groups.

The particle may comprise any suitable material including, for example, metals and/or metal alloys (e.g., tungsten carbide, iron oxide), polymers (e.g., polyesters, polyethers), ceramics, and silica. In some embodiments, the particle is associated with the polymeric material. In certain embodiments, the particle (e.g., the functionalized particle) may form a hydrogen bond with the polymeric material. In some embodiments, the particle may form a covalent bond with the polymeric material. In some such embodiments, the particle may be a polyfunctional monomer incorporated during the polymerization of the polymeric material (e.g., as a cross-linker).

The addition of a particle to the polymeric material may advantageously mitigate stress propagation (e.g., during mechanical deformation of the material such that cracking and/or breaking of the polymeric material is reduced as compared to polymeric materials without particles). In certain embodiments, the particle may comprise an active substance (e.g., a therapeutic agent encapsulated within the article).

In some embodiments the polymeric material is formed by the reaction of two or more polyfunctional monomers and an additional monomeric unit. In some embodiments, the additional monomeric unit comprises a compound containing one or more carboxylic acid derivatives. In some embodiments, the additional monomeric unit is a single compound containing at least one ester, amide or thioester group, or a mixture of compounds containing at least one ester, amide or thioester. In certain embodiments, the additional monomeric unit is a compound containing a lactone, lactam or thiolactone group. In certain embodiments, the additional monomeric unit is a naturally occurring lactone or lactam. In another embodiment, the additional monomeric unit lactone-containing or lactam-containing compound selected from the FDA's “Generally Recognized as Safe” Substances database and/or listed in 21 C.F.R. §182. In certain embodiments, the additional monomeric unit is selected γ-decalactone, δ-decalactone, ω-pentadecalactone, caprolactam, and mixtures thereof.

In certain embodiments of the invention, the additional monomeric unit does not contain a primary or secondary amine moiety.

In some embodiments, the molar ratio of the first polyfunctional monomer (e.g., comprising electrophilic reactive groups) to a mixture of additional polyfunctional monomers (e.g., comprising nucleophilic reactive groups) and/or additional monomeric units ranges between about 10:1 and about 1:10. In an exemplary embodiment, the molar ratio of the first polyfunctional monomer to a mixture of additional polyfunctional monomers and/or monomeric units is about 1:1. In certain embodiments, the molar ratio of first polyfunctional monomer to a mixture of additional polyfunctional monomers and/or monomeric units is at less than about 10:1, less than about 8:1, less than about 6:1, less than about 4:1, less than about 2:1, less than about 1.5:1, less than about 1:1, less than about 1.5:1, less than about 1:2, less than about 1:4, less than about 1:6, or less than about 1:8. In some embodiments, the molar ratio of first polyfunctional monomer to a mixture of additional polyfunctional monomers and/or monomeric units is greater than or equal to about 1:10, greater than or equal to about 1:8, greater than or equal to about 1:6, greater than or equal to about 1:4, greater than or equal to about 1:2, greater than or equal to about 1:1.5, greater than or equal to about 1:1, greater than or equal to about 1.5:1, greater than or equal to about 2:1, greater than or equal to about 4:1, greater than or equal to about 6:1, or greater than or equal to about 8:1. Combinations of the above-referenced ranges are also possible (e.g., between about 10:1 and about 1:10, between about 1:4 and about 4:1, between about 1:2 and about 2:1).

In some such embodiments, the second polyfunctional monomer is present in the mixture of additional polyfunctional monomers and/or monomeric units in an amount of at least about 10 mol %, at least about 20 mol %, at least about 25 mol %, at least about 50 mol %, at least about 75 mol %, at least about 90 mol %, or at least about 99 mol %. In certain embodiments, the second polyfunctional monomer is present in the mixture of additional polyfunctional monomers and/or monomeric units in an amount of less than or equal to about 99.9 mol %, less than or equal to about 99 mol %, less than or equal to about 90 mol %, less than or equal to about 75 mol %, less than or equal to about 50 mol %, less than or equal to about 25 mol %, or less than or equal to about 20 mol %. Combinations of the above-referenced ranges are also possible (e.g., between about 25 mol % and about 99.9 mol %). Other ranges are also possible.

In some embodiments, at least a portion of the electrophilic reactive groups and/or nucleophilic reactive groups are unreacted. For example, in some embodiments, at least about 1% of the reactive groups in the polymeric material, after forming the polymeric material, are free reactive groups. In some embodiments, at least about 0.05%, at least about 0.08%, at least about 1%, at least about 2%, at least about 5%, at least about 10%, at least about 20%, at least about 30%, at least about 33%, or at least about 40% of the reactive groups in the polymeric material are free reactive groups. In certain embodiments, less than or equal to about 50%, less than or equal to about 40%, less than or equal to about 33%, less than or equal to about 30%, less than or equal to about 20%, less than or equal to about 10%, less than or equal to about 5%, less than or equal to about 2%, or less than or equal to about 1% of the reactive groups in the polymeric material are free reactive groups. Combinations of the above referenced ranges are also possible (e.g., between about 0.05% and about 33%, between about 0.08% and about 50%, between about 20% and about 40%). Other ranges are also possible.

Those skilled in the art would understand that the free reactive groups described herein do not refer to the reactive groups participating during polymerization of the polymeric material, but to the free reactive groups present in the polymeric material after formation of the polymer (e.g., after heating and/or casting of the polymer, and cooling to room temperature, after thermal reconfiguration) as compared to the number of free reactive groups present in the mixture prior to the formation (i.e. polymerization) of the polymeric material. For example, in a particular embodiment, between about 1% and about 50% of the functional reactive groups present on the two or more polyfunctional monomers prior to formation of the polymeric material, are still available (i.e. free) for reacting after the polymerization (e.g., curing, baking, molding) of the polymeric material. The number of free reactive groups in the polymeric material may be substantially the same 1 hour, 5 hours, 24 hours, 48 hours, or 72 hours after polymerization of the material as compared to prior to polymerization of the material, as determined by the number of reactive groups (e.g., electrophilic reactive groups, nucleophilic reactive groups) present on the polyfunctional monomers.

In some embodiments, the thermal reconfiguration of the polymeric material is due to the presence of free reactive groups and internal transient bonds. For example, the polymeric material may be broken (e.g., ripped, torn, cut) and the free reactive groups may be capable of forming new bonds with other free reactive groups, and/or the free reactive groups may be capable of exchange with an already formed bond to establish a new bond, upon heating of the polymeric material (or pieces of polymeric material). In some embodiments, the polymeric material may be mechanically deformed and heated (e.g., above 90° C.) such that the free reactive groups form bonds with other free reactive groups and/or new free reactive groups are formed (i.e. bond exchange), such that upon cooling the polymeric material is thermally reconfigured into a new shape (as compared to the original shape of the material), as described above.

In some cases, the free reactive groups may be capable of forming internal associative reactions, such as hydrogen bonding with internal and external agents (e.g., as an adhesive). In certain embodiments, the free reactive groups may be capable of participating in pH-based reactions (e.g., buffering, ionic repulsion, salt formation) such as in pH responsive materials.

In some embodiments, the free reactive group is a carboxylic acid. In certain embodiments, the free reactive group comprises a carboxylic acid, a hydroxyl, an amine, a thiol, a hydroxyl, or an alkene capable of, for example, reacting with another free reactive group.

As described above, in some embodiments, two or more polyfunctional monomers are combined (i.e. reacted) in the presence of a catalyst.

In some embodiments, the catalyst is a nucleophile. In certain embodiments, the catalyst is a base (e.g., a mild base, a weak base). In certain embodiments, the catalyst is a metal salt. In some embodiments, the catalyst is a sulfate salt of zinc such as ZnSO₄ and hydrates thereof. An exemplary reaction in the presence of zinc sulfate is shown in FIG. 1A.

In some embodiments, the catalyst is selected from catalysts listed in FDA's “Generally Recognized as Safe” Substances database and/or listed in 21 C.F.R. §182. In certain embodiments, the catalyst is food grade and/or food derived catalyst.

In certain embodiments, the catalyst is an organic amine. In some embodiments, the catalyst is a tertiary amine. In some cases, the tertiary amine catalyst does not contain any amino N—H or NH₂ functional groups.

In some embodiments, the catalyst is an alkaloid compound. In certain embodiments, the catalyst is a purine base. Non-limiting examples of purine bases include purine, adenine, guanine, hypoxanthine, xanthine, theobromine, caffeine, uric acid and isoguanine. In an exemplary embodiment, the catalyst is caffeine. FIGS. 1B-1C are exemplary reactions in the presence of a caffeine catalyst, according to some embodiments.

As described above, the use of a food grade catalyst such as caffeine offers numerous advantages over traditional catalysts including FDA approval, low cytotoxicity, and/or a reduced need (or substantially no need) to remove the catalyst after polymerization.

In some embodiments, the catalyst (e.g., food grade catalyst) is present in the composition after the formation of the polymeric material in an amount ranging between 0.01 mol % and about 25 mol %. For example, in some embodiments, the composition comprises substantially no catalyst after the formation of the polymeric material. In certain embodiments, the catalyst is present in the composition after the formation of the polymeric material in an amount of at least about 0.01 mol %, at least about 0.05 mol %, at least about 0.1 mol %, at least about 0.5 mol %, at least about 1 mol %, at least about 2 mol %, at least about 5 mol %, at least about 10 mol %, or at least about 20 mol %. In certain embodiments, the catalyst is present in the composition after the formation of the polymeric material in an amount of less than or equal to about 25 mol %, less than or equal to about 20 mol %, less than or equal to about 10 mol %, less than or equal to about 5 mol %, less than or equal to about 2 mol %, less than or equal to about 1 mol %, less than or equal to about 0.5 mol %, less than or equal to about 0.1 mol %, or less than or equal to about 0.05 mol %. Combinations of the above-referenced ranges are also possible (e.g., between 1 mol % and 25 mol %, between 0.01 mol % and 5 mol %). Other ranges are also possible.

As described above, in some embodiments, the polymeric material may be formed using three or more polyfunctional monomers. FIG. 1D is an exemplary reaction scheme for a polymeric material formed by the reaction of four polyfunctional monomers in the presence of a catalyst (e.g., caffeine). In the exemplary reaction, polypropylene oxide is reacted with citric acid, mercaptosuccinic acid, and PPO-dimethacrylate in the presence of caffeine via Michael addition to form a branched polymeric material.

In certain embodiments, as described above, the polymeric material may be formed using two or more polyfunctional monomers and one or more additional monomeric units. FIG. 1E is an exemplary reaction scheme for a polymeric material formed by the reaction of two polyfunctional monomers and an additional monomeric unit in the presence of a catalyst. In the exemplary reaction, polypropylene oxide is reacted with citric acid in the presence of caffeine and caprolactone in the presence of triazabicyclodecene to form a dual polymer network material.

In some embodiments, the composition comprises a polymeric material, an additive associated with the polymeric material, and optionally a catalyst. The additive may be associated with (or incorporated into) the polymeric material by various means. For example, in some embodiments, the additive is covalently bound to the polymer backbone of the polymeric material. In certain embodiments, the additive is embedded within the polymeric material. In some cases, the additive is absorbed into the polymeric material after formation of the polymeric material. In certain embodiments, the additive is mixed with the two or more polyfunctional monomers (and optionally, the catalyst and/or additional monomeric units) before and/or during polymerization of the polymeric material.

In some embodiments, the presence of an additive in the polymeric material does not substantially inhibit the function of the additive.

In some embodiments, the additive is an active substance, which can be a therapeutic, nutraceutical, prophylactic or diagnostic agent, an herbicide, fertilizer, insecticide, insect repellent, or other material of similar nature. The active substance may be entrapped within the polymeric material or may be directly attached to one or more atoms in the polymeric material through a chemical bond. Representative bond types include covalent and ionic. In certain embodiments, the active substance is covalently bonded to the polymeric material. In some embodiments, the active substance is bonded to the polymeric material through a carboxylic acid derivative. In some cases, the carboxylic acid derivative may be an ester bond.

Active substances that contain a carboxylic acid group may be directly incorporated into polymeric materials that contain ester and hydroxyl groups without further modification. Active substances containing an alcohol may first be derivatized as a succinic or fumaric acid monoester and then incorporated into the polymeric material. Active substances that contain a thiol may be incorporated into olefin or acetylene-containing materials through a sulfur-ene reaction. In other embodiments, the one or more agents are non-covalently associated with the polymeric material (e.g., dispersed or encapsulated within).

In certain embodiments, the composition is constructed and arranged to release the therapeutic agent from the polymeric material. In certain embodiments, the active substance is designed to be released from the polymeric material. Such embodiments may be useful in the context of drug delivery. In other embodiments, the active substance is permanently affixed to the polymeric material. Such embodiments may be useful in molecular recognition and purification contexts.

In some embodiments, the active substance is a radiopaque material such as tungsten carbide or barium sulfate.

In certain embodiments, the active substance is a therapeutic agent. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Therapeutic agents include, without limitation, agents listed in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005. In some embodiments, the therapeutic agent may be selected from “Approved Drug Products with Therapeutic Equivalence and Evaluations,” published by the United States Food and Drug Administration (F.D.A.) (the “Orange Book”). In some cases, the therapeutic agent is one that has already been deemed safe and effective for use in humans or animals by the appropriate governmental agency or regulatory body. For example, drugs approved for human use are listed by the FDA under 21 C.F.R. §§330.5, 331 through 361, and 440 through 460, incorporated herein by reference; drugs for veterinary use are listed by the FDA under 21 C.F.R. §§500 through 589, incorporated herein by reference. All listed drugs are considered acceptable for use in accordance with the present invention. In certain embodiments, the therapeutic agent is a small molecule. Exemplary classes of agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents (e.g., taxanes, such as paclitaxel and docetaxel; cisplatin, doxorubicin, methotrexate, etc.), antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated. These may be vitamins, supplements such as calcium or biotin, or natural ingredients such as plant extracts or phytohormones.

In some embodiments, the therapeutic agent is one or more antimalarial drugs. Exemplary antimalarial drugs include quinine, lumefantrine, chloroquine, amodiaquine, pyrimethamine, proguanil, chlorproguanil-dapsone, sulfonamides such as sulfadoxine and sulfamethoxypyridazine, mefloquine, atovaquone, primaquine, halofantrine, doxycycline, clindamycin, artemisinin and artemisinin derivatives. In some embodiments, the antimalarial drug is artemisinin or a derivative thereof. Exemplary artemisinin derivatives include artemether, dihydroartemisinin, arteether and artesunate. In certain embodiments, the artemisinin derivative is artesunate.

Non-limiting examples of therapeutic agents are shown in FIG. 1G. In a particular embodiment, the therapeutic agent is ivermectin.

In another embodiment, the therapeutic agent is an immunosuppressive agent. Exemplary immunosuppressive agents include glucocorticoids, cytostatics (such as alkylating agents, antimetabolites, and cytotoxic antibodies), antibodies (such as those directed against T-cell recepotors or 11-2 receptors), drugs acting on immunophilins (such as cyclosporine, tacrolimus, and sirolimus) and other drugs (such as interferons, opioids, TNF binding proteins, mycophenolate, and other small molecules such as fingolimod).

In a further embodiment, the active substance is used to prevent restenosis in a drug-eluting stent. Exemplary agents include sirolimus (rapamycin), everolimus, zotarolimus, biolimus A9, cyclosporine, tranilast, paclitaxel and docetaxel.

In a further embodiment, the active substance is an antimicrobial agent. Exemplary antimicrobials include antibiotics such as aminoglycosides, cephalosporins, chloramphenicol, clindamycin, erythromycins, fluoroquinolones, macrolides including fidaxomicin and rifamycins such as rifaximin, azolides, metronidazole, penicillins, tetracyclines, trimethoprim-sulfamethoxazole, oxazolidinone such as linezolid, and glycopeptides such as vancomycin. Other antimicrobial agents include antifungals such as antifungal polyenes such as nystatin, amphotericin, candicidin and natamycin, antifungal azoles, allylamine antifungals and echinocandins such as micafungin, caspofungin and anidulafungin.

In some embodiments, the therapeutic agent is a small molecule drug having molecular weight less than about 2500 Daltons, less than about 2000 Daltons, less than about 1500 Daltons, less than about 1000 Daltons, less than about 750 Daltons, less than about 500 Daltons, less or than about 400 Daltons. In some cases, the therapeutic agent is a small molecule drug having molecular weight between 200 Daltons and 400 Daltons, between 400 Daltons and 1000 Daltons, or between 500 Daltons and 2500 Daltons.

In other embodiments, the active substance is a protein or other biological macromolecule. Such substances may be covalently bound to the polymeric material through ester bonds using available carboxylate containing amino acids, or may be incorporated into polymeric material containing olefinic or acetylenic moieties using a thiol-ene type reaction. In some cases, the active substance comprises an amine functional group capable of reacting with an epoxide functional group (e.g., on a polyfunctional monomer) to form an amide or ester bond. In other embodiments, the active substance is non-covalently associated with the polymeric material. In some such embodiments, the active substance may be dispersed or encapsulated within by hydrophilic and/or hydrophobic forces.

In some embodiments, as described herein, the additive may be loaded before formation of the polymeric material. Such loading permits incorporation (e.g., trapping) of relatively large molecules that otherwise couldn't be loaded (e.g., in traditional thermosets and/or crosslinked polymeric materials). For example, in some embodiments, the additive (e.g., the therapeutic agent) is crystalline or semicrystalline. The crystalline (or semicrystalline) additive may be added, for example, during polymerization of the polymeric material such that the crystalline (or semicrystalline) additive may be associated with the polymeric material. The polymeric materials described herein advantageously permit the incorporation of crystalline materials otherwise not possible (e.g., due to required melting of the crystalline materials in traditional polymeric materials). For example, the additive may be a crystalline or semicrystalline therapeutic agent. In some cases, the additive comprises a therapeutic agent conjugated to a macromolecule or a particle.

In certain embodiments, the additive (e.g., the crystalline or semicrystalline additive) has a largest cross-sectional dimension greater than or equal to an average pore size of the polymeric material (e.g., an average pore size of the polymeric material in the absence of the additive). In some embodiments, the additive is dispersed homogeneously within the polymeric material. For example, as compared to traditional crosslinked polymeric materials in which the additive may be absorbed into the polymeric material after formation of the polymeric material, which may result in a heterogeneous distribution of the additive within the polymeric material, the polymeric materials described herein may permit homogeneous dispersion of the additive within the polymeric material. In some cases, the additive is dispersed heterogeneously within the polymeric material.

The additive (e.g., active substance) may be associated with the polymeric material and present in the composition in any suitable amount. In some embodiments, the additive is present in the composition an amount ranging between about 0.01 wt % and about 50 wt % versus the total composition weight. In some embodiments, the additive is present in the composition in an amount of at least about 0.01 wt %, at least about 0.05 wt %, at least about 0.1 wt %, at least about 0.5 wt %, at least about 1 wt %, at least about 2 wt %, at least about 3 wt %, at least about 5 wt %, at least about 10 wt %, at least about 20 wt %, at least about 30 wt %, at least about 40 wt % versus the total composition weight. In certain embodiments, the additive is present in the composition in an amount of less than or equal to about 50 wt %, less than or equal to about 40 wt %, less than or equal to about 30 wt %, less than or equal to about 20 wt %, less than or equal to about 10 wt %, less than or equal to about 5 wt %, less than or equal to about 3 wt %, less than or equal to about 2 wt %, less than or equal to about 1 wt %, less than or equal to about 0.5 wt %, less than or equal to about 0.1 wt %, or less than or equal to about 0.05 wt %. Combinations of the above-referenced ranges are also possible (e.g., between about 0.01 wt % and about 50 wt %). Other ranges are also possible.

Advantageously, the polymeric materials described herein may permit higher concentrations (weight percents) of active substances such as therapeutic agents to be incorporated into the polymeric material as compared to other polymers such as hydrogels.In some embodiments, the additive (e.g., the active substance) may be released from the polymeric material. In certain embodiments, the additive is released by diffusion out of the polymeric material. In some embodiments, the additive is released by degradation of the polymeric material (e.g., biodegradation, enzymatic degradation, hydrolysis). In some embodiments, the additive (e.g., active substance) is released from the composition at a particular rate. In some embodiments, between 0.05% to 0.99% of the active substance is released between 1 minute and 1 year. In some embodiments, between about 0.05 vol % and about 99.0 vol % of the active substance is released from the polymeric material after a certain amount of time. In some embodiments, at least about 0.05 vol %, at least about 0.1 vol %, at least about 0.5 vol %, at least about 1 vol %, at least about 5 vol %, at least about 10 vol %, at least about 20 vol %, at least about 50 vol %, at least about 75 vol %, at least about 90 vol %, at least about 95 vol %, or at least about 98 vol % of the active substance associated with the polymeric material is released from the composition after about 1 minute, after about 5 minutes, after about 20 minutes, after about 1 hour, after about 2 hours, after about 5 hours, after about 10 hours, after about 24 hours, after about 32 hours, after about 72 hours, after about 96 hours, or after about 192 hours. In certain embodiments, at least about 0.05 vol %, at least about 0.1 vol %, at least about 0.5 vol %, at least about 1 vol %, at least about 5 vol %, at least about 10 vol %, at least about 20 vol %, at least about 50 vol %, at least about 75 vol %, at least about 90 vol %, at least about 95 vol %, or at least about 98 vol % of the active substance associated with the polymeric material is released from the composition after about 1 day, after about 5 days, after about 30 days, after about 60 days, after about 120 days, or after about 365 days. For example, in some cases, at least about 90 vol % of the active substance associated with the polymeric material is released from the composition after about 120 days.

As mentioned, in some embodiments, the composition includes an active substance associated with the polymeric material. In some cases, the active substance is associated with the polymeric material by being arranged directly adjacent (e.g., in contact with) the backbone of the polymeric material. In certain embodiments, the active substance is embedded within the polymeric material. In some embodiments, the active substance is associated with the polymeric material via formation of a bond, such as an ionic bond, a covalent bond, a hydrogen bond, Van der Waals interactions, and the like. The covalent bond may be, for example, carbon-carbon, carbon-oxygen, oxygen-silicon, sulfur-sulfur, phosphorus-nitrogen, carbon-nitrogen, metal-oxygen, or other covalent bonds. The hydrogen bond may be, for example, between hydroxyl, amine, carboxyl, thiol, and/or similar functional groups.

In some embodiments, the active substance is covalently bound to the backbone of the polymeric material. In some embodiments, the active substance is Incorporated within the backbone of the polymeric material. For example, in some embodiments, the active substance is present during polymerization of the two or more polyfunctional monomers, such that the active substance covalently binds to one or more of the polyfunctional monomers (e.g., a reactive functional group on the active substance reacts with at least one reactive functional group on the one or more polyfunctional monomers).

The release rate of the additive may be controlled (i.e. tuned) by changing the selection and properties of polyfunctional monomers, ratio of the monomers, reactive groups, or the like. In some cases, the release rate of the additive can be modulated by texturing a surface of the polymeric material such that water accessibility to the surface of the material is reduced (e.g. as compared to an untextured surface of the polymeric material). For example, the texture of the surface of polymeric materials can be tuned via molding of the polymeric material. For example, a more hydrophobic surface can be imparted through the use of lotus leaf patterning, slowing the absorption of aqueous media. Alternatively, in some cases, the addition of humectants as an additive to the polymeric material may enhance the rate of absorption and change release kinetics. In some embodiments, coatings (e.g., an enteric coating such as shellac) deposited on the polymeric material can be used to tune release rate by allowing permeation of liquid in certain environments.

In some cases, the partition coefficient of the active substance in the polymeric material can be tuned. For example, if the active substance is hydrophobic, a hydrophobic polymeric material backbone may, in some cases, slow the release into aqueous solution, however, a hydrophilic polymeric material backbone should accelerate it. Additionally, a hydrophilic polymeric material backbone may, in some cases, increase the rate of water absorption into the material, expanding (e.g., swelling) the polymeric material and accelerating release rate. The expansion and dissolution of the material may be increased, in some embodiments, under conditions when free reactive groups contain ionizable moieties that become charged in the presence of aqueous media. In some such embodiments, as the material disintegrates due to ionic repulsion, the rate of release of contents may be increased via diffusion and/or better access to cleavable bonds may be imparted. Those skilled in the art would be capable of selecting suitable methods for determining the partition coefficient of the active substance including, for example, high performance liquid chromatography (HPLC).

In some embodiments, the active substance is a polyfunctional monomer, as described above. In an exemplary embodiment, the polymeric material comprises a first polyfunctional monomer comprising polyethylene glycol and/or propylene glycol and a second polyfunctional monomer comprising citric acid as the active substance. In some such embodiments, the active substance may be released from the composition upon biodegradation of the polymeric material.

In some embodiments, the composition comprises a first polymeric material and a second polymeric material. The first polymeric material and second polymeric material may be the same or different and comprise a polymer having the structure as in Formula (I) and are formed by the reaction of a first polyfunctional monomer and a second polyfunctional monomer. The first polymeric material and second polymeric material may be, in some cases, entangled. In certain embodiments, the first polymeric material comprises the structure as in Formula (I) formed by the reaction of a first polyfunctional monomer and a second polyfunctional monomer, and the second polymeric material is a macromolecule. In certain embodiments, the macromolecule entangles (e.g., chain entanglement) with the first polymeric material. The macromolecule may comprise any suitable material including, for example, polymers, co-polymers, and/or carbohydrates (e.g. such as starches or polysaccharides). Non-limiting examples of suitable materials include natural polymers such as silk, carbohydrates such as tapioca root and arrowroot, synthetic polymers such as polymethylmethacrylate and polydimethylsiloxane (e.g., polydimethylsiloxane-g-acrylates), polyoxamers such as pluronic, or the like. Incorporation of macromolecules into the composition may offer several advantages including, for example, the ability to tune the mechanical properties of the composition by selecting certain macromolecules (e.g., increased toughness, increased Young's elastic modulus), stabilize active substances associated with the polymeric material, and/or provide surfactant-like properties to the polymeric material. Those skilled in the art would be capable of selecting suitable macromolecules for incorporation into the composition, based upon the teachings of the specification. The macromolecules may be added, for example, before and/or during polymerization of the polymeric material.

In some cases, the composition may also contain other additives, such as plasticizers, stabilizers, preservatives, antioxidants, dyes, pigments, flavoring agents, or the like.

In some embodiments, the composition comprises a polymeric material, an additive (e.g., active substance), and, optionally, a food-grade catalyst. In certain embodiments, the composition comprises substantially no auxiliary materials other than the crosslinked polymeric material, additive (e.g., active substance), and catalyst. In some embodiments, the composition comprises less than about 10 wt %, less than about 8 wt %, less than about 5 wt %, less than about 3 wt %, less than about 2 wt %, or less than about 1 wt % auxiliary materials. Combinations of the above-referenced ranges are also possible (e.g., between about 1 wt % and about 10 wt % auxiliary materials).

Auxiliary materials include, for example, solvents, water, non-food grade catalysts, non-FDA approved materials, and/or excipients. In some cases, auxiliary materials may include toxic compounds (e.g., cytotoxic). The term “toxic” refers to a substance showing detrimental, deleterious, harmful, or otherwise negative effects on a subject, tissue, or cell when or after administering the substance to the subject or contacting the tissue or cell with the substance, compared to the subject, tissue, or cell prior to administering the substance to the subject or contacting the tissue or cell with the substance. In certain embodiments, the effect is death or destruction of the subject, tissue, or cell. In certain embodiments, the effect is a detrimental effect on the metabolism of the subject, tissue, or cell. In certain embodiments, a toxic substance is a substance that has a median lethal dose (LD50) of not more than 500 milligrams per kilogram of body weight when administered orally to an albino rat weighing between 200 and 300 grams, inclusive. In certain embodiments, a toxic substance is a substance that has an LD50 of not more than 1,000 milligrams per kilogram of body weight when administered by continuous contact for 24 hours (or less if death occurs within 24 hours) with the bare skin of an albino rabbit weighing between two and three kilograms, inclusive. In certain embodiments, a toxic substance is a substance that has an LC50 in air of not more than 2,000 parts per million by volume of gas or vapor, or not more than 20 milligrams per liter of mist, fume, or dust, when administered by continuous inhalation for one hour (or less if death occurs within one hour) to an albino rat weighing between 200 and 300 grams, inclusive. The term “non-toxic” refers to a substance that is not toxic. Toxic compounds include, e.g., oxidative stressors, nitrosative stressors, proteasome inhibitors, inhibitors of mitochondrial function, ionophores, inhibitors of vacuolar ATPases, inducers of endoplasmic reticulum (ER) stress, and inhibitors of endoplasmic reticulum associated degradation (ERAD). In some embodiments a toxic agent selectively causes damage to nervous system tissue. Toxic compounds include compounds that are directly toxic and agents that are metabolized to or give rise to substances that are directly toxic. It will be understood that the term “toxic compounds” typically refers to compounds that are not ordinarily present in a cell's normal environment at sufficient levels to exert detectable damaging effects. However, in some cases, the toxic compounds may be present in a cell's normal environment but at concentrations significantly less than present in the auxiliary materials described herein. Typically toxic compounds exert damaging effects when present at a relatively low concentration, e.g., at or below 1 mM, e.g., at or below 500 microM, e.g., at or below 100 microM. It will be understood that a toxic agent typically has a threshold concentration below which it does not exert detectable damaging effects. The particular threshold concentration will vary depending on the agent and, potentially, other factors such as cell type, other agents present in the environment, etc.

In some embodiments, the composition comprises substantially no solvent. In certain embodiments, the composition comprises the polymeric material, a therapeutic agent, and substantially no additional materials other than those included on the FDA's “Generally Recognized as Safe” Substances database and/or listed in 21 C.F.R. §182.

Advantageously, the compositions and polymeric materials described herein may not undergo side reactions (i.e. undesired reactions) with other reactive groups and/or materials due to, for example, the lack of auxiliary materials in the composition.

In some embodiments, the composition is prepared by combining two or more polyfunctional monomers at a temperature before the gel point. The catalyst, additional monomeric unit, active substance and other additives may be added as needed and reacted to reach the gel point. The two or more polyfunctional monomers and other ingredients (e.g., catalysts, additional polyfunctional monomers, additional monomeric units, additives) may be combined at a particular temperature. In some embodiments, the two or more polyfunctional monomers and other ingredients are combined at between 20° C. and 90° C. In certain embodiments, the two or more functional monomers and other ingredients are combined at a temperature of at least about 20° C., at least about 40° C., at least about 60° C., at least about 70° C., or at least about 80° C. In certain embodiments, the two or more polyfunctional monomers and other ingredients are combined at a temperature less than or equal to about 90° C., less than or equal to about 80° C., less than or equal to about 70° C., less than or equal to about 60° C., less than or equal to about 40° C., or less than or equal to about 25° C. Combinations of the above-referenced ranges are also possible (e.g., between 20° C. and 90° C., between 20° C. and 25° C., between 25° C. and 70° C., between 70° C. and 90° C.). In some cases, the mixture of polyfunctional monomers and other ingredients may be stirred before and/or during polymerization. Those skilled in the art would be capable of selecting suitable methods for mixing the composition. Generally, crosslinking density is increased as a function of baking time.

Advantageously, the polymeric material may be formed, in some embodiments, in a single step. For example, two or more polyfunctional monomers, one or more catalysts, optionally one or more additives, optionally, one or more monomeric units, and optionally, one or more macromolecules, may be mixed together and the polymeric material may be formed upon heating of the mixture to a temperature such that the polymeric material polymerizes (e.g., at least about 30° C., at least about 60° C., at least about 70° C., at least about 90° C.).

After a period of time, the mixture may be poured into a mold and incubated at at a particular temperature, such as 90° C. In certain embodiments, the incubation temperature may be at least about 40° C. at least about 60° C., at least about 80° C., or at least about 90° C. In some embodiments, the incubation temperature may be less than or equal to about hundred and 20° C., less than or equal to about hundred ° Celsius, less than or equal to about 90° C., less than or equal to about 80° C., or less than or equal to about 60° C. Other incubation temperatures are also possible. In some embodiments, after cooling, the polymeric material may be separated from the mold. The mold may comprise any suitable size and/or shape. For example, in some embodiments, the mold comprises an external housing such as a straw, tubing, or the like. In certain embodiments, the polymeric material may be retained in the mold (e.g., in the manufacture of device comprising the mold and the polymeric material).

In certain embodiments, the composition and/or polymeric material is characterized by a Young's modulus between 0.01 and 500.00 N/mm², between 0.01 and 100.00 N/mm², more between 0.01 and 50.00 N/mm², between 0.01 and 10.00 N/mm², or between 0.01 and 5.00 N/mm². The Young's Modulus can be evaluated through mechanical testing such as compressive or tensile testing. Those skilled in the art would be capable of selecting method for determining the Young's modulus including for example, using an Instron in tensile mode with uniaxial loading, testing a cast necked or dog-bone shaped sample, according to ASTM D412. The Young's Modulus may be evaluated by calculating the gradient of the linear region of the Stress-Strain graph, where Young's Modulus E=/c. In certain embodiments, the composition and/or polymeric material is characterized by a Young's modulus between 0.01-0.1 N/mm². In other embodiments, the Young's Modulus is between 0.1 and 3.0 N/mm², between 0.2 and 2.0 N/mm², or between 0.3 and 1.5 N/mm².

Some embodiments, the composition and/or polymeric material is characterized by a compression modulus between 0.05 N/mm² and 20 N/mm², between 0.1 N/mm² and 10 N/mm², or between 0.5 N/mm² and 20 N/mm². Those skilled in the art would be capable of selecting method for determining the compression modulus including for example, using an Instron in compression mode with uniaxial loading, testing a cast cylindrical sample, according to ASTM D412.

In certain embodiments, the composition and/or polymeric material is characterized by a tensile strength between 0.01 N/mm² and 5.00 N/mm². The tensile strength of a composition and/or polymeric material may be determined, in some embodiments, by measuring the force required to break a material extended in a unilateral direction by using an instrument such as an Instron to calculate force required to break a standardized shape such as a dogbone shaped material, according to ASTM D575. In certain embodiments, the composition and/or polymeric material is characterized by a tensile strength between 0.03 and 5.00 N/mm², between 0.05 and 3.00 N/mm², or between 0.1 and 1.00 N/mm².

In some embodiments, the composition and/or polymeric materials characterized by a shear modulus between 0.02 N/mm² and 9 N/mm². The shear modulus of a composition and/or polymeric material may be determined, in some embodiments, by sheer rheometry, according to ASTM D7605.

In certain embodiments, the composition and/or polymeric material is characterized by a crosslinking density between 1 and 550 mol/m³. The crosslinking density of a composition and/or polymeric material may be determined by using the formula n=E/3RT, where E is the Young's Modulus evaluated from the tensile test, R is the ideal gas constant and T is temperature (298 K). In certain embodiments, the composition and/or polymeric material is characterized by a crosslinking density between 5-550 mol/m³, between 65-550 mol/m³, between 65-300 mol/m³, between 100-300 mol/m³, between 200-400 mol/m³, 5-70 mol/m³, between 1-15 mol/m³, between 10-75 mol/m³, between 10-65 mol/m³, between 20-60 mol/m³, or between 30-50 mol/m³.

In certain embodiments, the composition and/or polymeric material is capable of absorbing water or solvent without dissolution or significant loss of topography, i.e., forms a hydrogel. In certain embodiments, the composition and/or polymeric material is capable of absorbing water or solvent in an amount that is at least 1%, 5%, 10%, 15%, 20%, 25%, 30%, 35%, 40%, 45%, 50%, 55%, 60%, 65%, 70%, 75%, 80%, 85%, 90%, 95%, or 100% or more by weight. In certain embodiments, the composition and/or polymeric material is capable of absorbing water or solvent in an amount that is at least 500% by weight. In other embodiments, the composition and/or polymeric material is capable of absorbing water or solvent in an amount between 100-800% by weight, such as 100%, 150%, 200%, 250%, 300%, 350%, 400%, 450%, 500%, 550%, 600%, 650%, 700%, 750%, or 800%. The absorption capability of the composition and/or polymeric material may be determined by swelling studies that evaluate the mass of a given material before and after incubation in solution.

The Young's modulus, tensile strength, absorption, and/or crosslinking density may be controlled (i.e. tuned) by changing the selection and properties of polyfunctional monomers, ratio of the monomers, reactive groups, or the like.

In certain embodiments, the composition and/or polymeric material is characterized in that it retains its shape at temperatures above 23° C. By way of example, a gravity test can be used to establish whether a particular polymeric material will retain its shape. In particular, a 1×1 cm² cylinder of the composition and/or polymeric material may be prepared, and left to stand for 24 hours at 50° C. under a standard atmosphere. In certain embodiments, the composition and/or polymeric material will lose less than 20% of its height over this time, and in other embodiments, the composition and/or polymeric material will lose less than 5% of its height over this time.

In certain embodiments, the composition and/or polymeric material is characterized in that it will not dissolve in common solvents, such as solvents that are capable of dissolving the monomeric components. By way of example, a 1×1 cm² cylinder of a composition and/or polymeric material may be prepared, and left to stand in 15 ml of solvents such as DMSO, acetonitrile or water for 24 hours at either 23° C. or 37° C. In certain embodiments, no dissolution of the composition and/or polymeric material is observed over this time period.

The compositions described herein advantageously may be capable of undergoing sterilization such as autoclaving and/or ultraviolet radiation without substantial changes to the mechanical properties and/or shape to the composition. For example, the ability of the composition to be sterilized non-destructively may be a result, in some cases, of the thermal reconfigurability of the composition.

As described herein, the composition may have dynamic properties such as the ability to undergo thermal reconfiguration.

Devices and articles comprising the compositions and/or polymeric materials are described below.

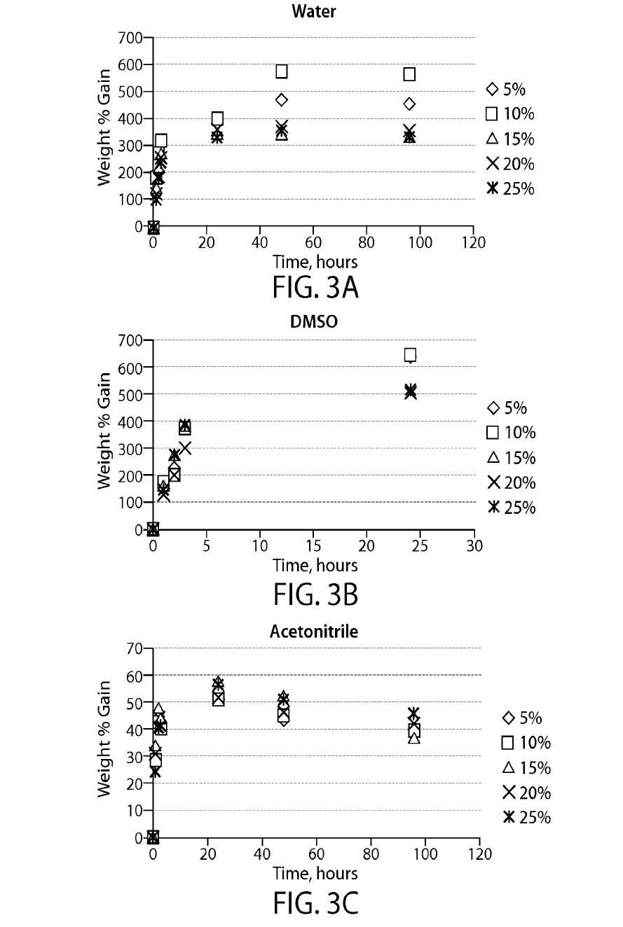

Examples of articles manufactured by molding the composition comprising the polymeric material are depicted in FIG. 3. In an exemplary embodiment (FIG. 3), each of the rings is loaded with 480 mg of artesunate. In some embodiments, the introduction of an active substance into the polymeric material does not compromise the mechanical integrity of the polymeric material nor the ability to form complex shapes using the polymeric material.

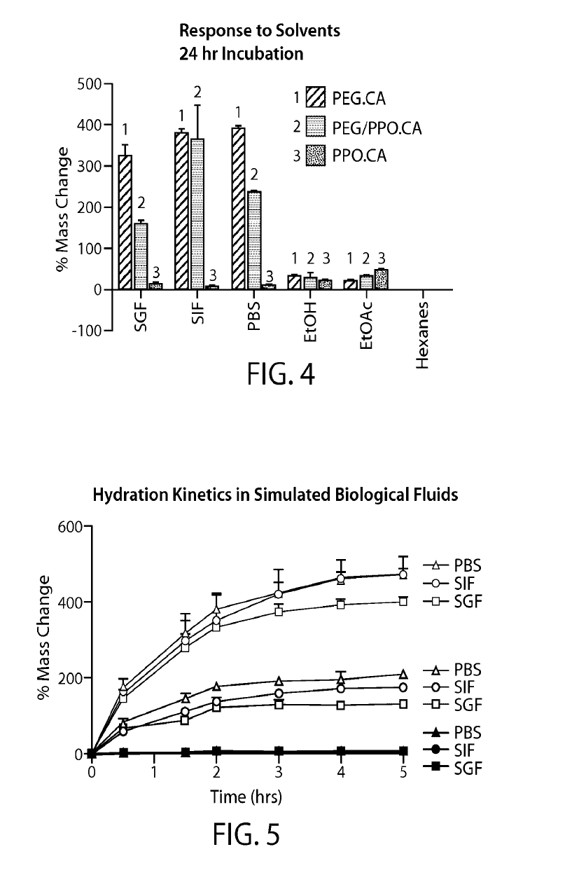

Articles made from the polymeric material may be manipulated in a variety of ways. For example, rectangular flat blocks of Example 1 were prepared by placing the polymeric material in a horseshoe-shaped fixture (FIGS. 4A-B) and incubating at 90° C. for varying time points (t=0-20 hours). The flat rectangular blocks in FIGS. 4A-B were suspended parallel to the ground and photographed on edge (FIG. 4C).

In addition to bending, the polymeric material may also be rolled and twisted (FIG. 5). In some embodiments, the polymeric material is formed (e.g., polymerized) in a mold having a particular shape, dimension, and/or contour. In certain embodiments, the polymeric material may be thermally reconfigured to obtain a new shape, dimension, and/or contour. In some embodiments, the polymeric material may be mechanically deformed (e.g., twisted, rolled, compressed, stretched, bent, curled, wrinkled, etc.) and incubated at a particular temperature (e.g., at or above about 90° C.) such that the polymeric material maintains the new shape. In certain embodiments, the polymeric material may have a first shape type (e.g., circular, square, rectangular, oval), first dimension (e.g., cross-sectional dimension), and/or first contour (e.g., planar) as defined by the surface with the largest surface area and a second shape after thermal reconfiguration, different than the first shape in type (e.g., such as circular, square, rectangular, oval), dimension (e.g., shorter, longer, thicker, thinner, etc.), and/or contour (e.g., curled, twisted, bent, etc.) such that the material maintains the new shape, dimension, and/or contour upon cooling (e.g., to room temperature (e.g., between about 20° C. and about 25° C.)).

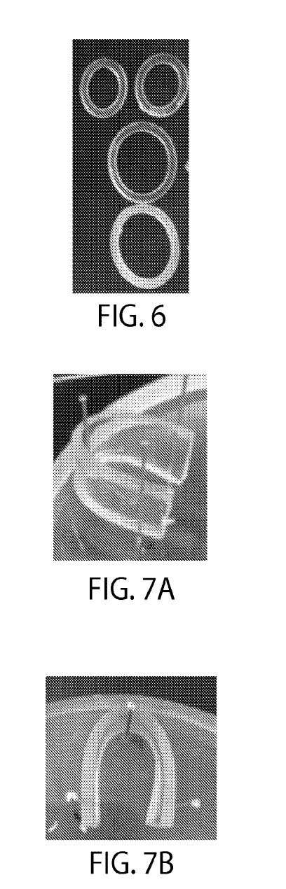

Separate articles made from the polymeric material may be joined together in the presence of heat. In one embodiment, a fractured article may be repaired as depicted in FIG. 6. In another embodiment, two separate articles may be joined together to construct an article which is not easily obtained from a single mold. In some embodiments a first article and a second article comprising the same polymeric material may be joined, wherein the polymeric material is as described herein. In certain embodiments, a first article comprising a first polymeric material and a second article comprising a second polymeric material, different than the first polymeric material, may be joined, wherein the polymeric materials are as described herein. In some embodiments, the fusion between the separate articles has a mechanical strength similar to an individually molded article.

Medical devices (e.g., implants) fabricated using polymeric materials described herein have several advantages. For example, the medical devices (e.g., implants) may be made directly in a molding process, or polymeric material stock may be produced that can be machined, cut, drilled, or otherwise converted into the desired device. In contrast to conventional thermosets, which can generally only be shaped by removing material, different pieces of the polymeric material described herein may also be joined together, permitting the construction of complex devices and machines.

In a further embodiment, the polymeric material is used to fabricate medical devices. For example, the polymeric material may be used to make partially or fully absorbable biocompatible medical devices, or components thereof. In some cases, the device to be fabricated is dependent on the mechanical properties of the polymeric material. For example, polymeric materials that are elastic/flexible may be used to form devices that require such properties to be effective. Elastic and flexible materials are typically those which have a lower degree of crosslinking, which can be achieved by controlling, for example, the bake time of the polymeric material, the polyfunctional monomers reacted, and/or the ratio of two or more polyfunctional monomers. In some cases, elastic and flexible properties may be imparted by the incorporation of additional polymers into the polymeric material, as described above (e.g., silk) and/or additives, as described herein.

Devices comprising the polymeric materials described herein include but are not limited to, sutures, barbed suture, braided suture, monofilament suture, hybrid suture of monofilament and multifilament fibers, braids, ligatures, knitted or woven meshes, knitted tubes, catheters, monofilament meshes, multifilament meshes, patches, wound healing device, bandage, wound dressing, burn dressing, ulcer dressing, skin substitute, hemostat, tracheal reconstruction device, organ salvage device, dural substitute, dural patch, nerve guide, nerve regeneration or repair device, hernia repair device, hernia mesh, hernia plug, device for temporary wound or tissue support, tissue engineering scaffold, guided tissue repair/regeneration device, anti-adhesion membrane, adhesion barrier, tissue separation membrane, retention membrane, sling, device for pelvic floor reconstruction, urethral suspension device, device for treatment of urinary incontinence, device for treatment of vesicoureteral reflux, bladder repair device, sphincter muscle repair device, injectable particles, injectable microspheres, bulking or filling device, bone marrow scaffold, clip, clamp, screw, pin, nail, medullary cavity nail, bone plate, interference screw, tack, fastener, rivet, staple, fixation device for an implant, bone graft substitute, bone void filler, suture anchor, bone anchor, ligament repair device, ligament augmentation device, ligament graft, anterior cruciate ligament repair device, tendon repair device, tendon graft, tendon augmentation device, rotator cuff repair device, meniscus repair device, meniscus regeneration device, articular cartilage repair device, osteochondral repair device, spinal fusion device, device for treatment of osteoarthritis, viscosupplement, stent, including coronary, cardiovascular, peripheral, ureteric, urethral, urology, gastroenterology, nasal, ocular, or neurology stents and stent coatings, stent graft, cardiovascular patch, catheter balloon, vascular closure device, intracardiac septal defect repair device, including but not limited to atrial septal defect repair devices and PFO (patent foramen ovale) closure devices, left atrial appendage (LAA) closure device, pericardial patch, vein valve, heart valve, vascular graft, myocardial regeneration device, periodontal mesh, guided tissue regeneration membrane for periodontal tissue, ocular cell implant, imaging device, cochlear implant, embolization device, anastomosis device, cell seeded device, cell encapsulation device, controlled release device, drug delivery device, plastic surgery device, breast lift device, mastopexy device, breast reconstruction device, breast augmentation device (including devices for use with breast implants), breast reduction device (including devices for removal, reshaping and reorienting breast tissue), devices for breast reconstruction following mastectomy with or without breast implants, facial reconstructive device, forehead lift device, brow lift device, eyelid lift device, face lift device, rhytidectomy device, thread lift device (to lift and support sagging areas of the face, brow and neck), rhinoplasty device, device for malar augmentation, otoplasty device, neck lift device, mentoplasty device, cosmetic repair device, and device for facial scar revision.

In a further embodiment, the medical device is fabricated from a polymeric material having one or more active substances. In one embodiment, the active substance is a therapeutic agent which can reduce pain and/or inflammation, enhance device attachment in the body, or reduce the likelihood of infection or device rejection. In a further embodiment, the device is a stent and the active substance is an agent that prevents restenosis. In another embodiment, the device is an implantable article and the active substance is an agent for the prevention or suppression of implant rejection and/or promote inflammation to achieve intentional fibrosis for cosmetic purposes.

In another embodiment, a drug-delivery carrier is fabricated from the polymeric material. Non-limiting examples of drug-delivery carriers include those for oral, rectal, vaginal and transdermal administration. The release rate of the active substance from the carrier as well as the degradation rate of the carrier itself may be adjusted depending on the particular monomer units used to prepare the polymeric material. Techniques for preparing such forms are known in the art.

In some embodiments, the compositions described herein may be used as a tissue adhesive. For example, the compositions may adhere to a particular type of human tissue (e.g., mucus membranes such as the lining of the gastric environment). The strength of adhesion between the composition comprising the polymeric material and human tissue may be between about 0.1 N/cm² and about 1 N/cm². In some embodiments, the strength of adhesion is at least about 0.1 N/cm², at least about 0.2 N/cm², at least about 0.4 N/cm², at least about 0.6 N/cm², or least about 0.8 N/cm². In certain embodiments, the strength of adhesion is less than or equal to about 1 N/cm², less than or equal to about 0.8 N/cm², less than or equal to about 0.6 N/cm², less than or equal to about 0.4 N/cm², or less than or equal to about 0.2 N/cm². Combinations of the above-referenced ranges are also possible (e.g., between about 0.1 N/cm² and about 1 N/cm²). Other ranges are also possible. The strength of adhesion may be measured by, for example, compressing the composition into excised tissue in an Instron for 5 minutes at 0.5 N, and measuring the force required to detach the composition from the tissue.

In certain embodiments, the compositions described herein may be used for taste masking. In some such embodiments, an active substance (e.g., a therapeutic agent) which generally has an unsavory taste may be incorporated into the compositions described herein in order to mask the unsavory taste of the active substance (e.g., when delivered orally). In certain embodiments, the compositions comprise polyfunctional monomers and/or additional monomeric units comprising odorants and/or flavors known in the food industry (e.g., such as lactones).

As described herein, the compositions and/or polymeric materials may be molded to have a particular shape. In certain embodiments, the compositions and/or polymeric materials may be molded to have a particular texture. For example, in some embodiments, the surface of the composition and/or polymeric material may be rough and/or have particular features which offer advantageous properties as compared to thermosetting materials. In certain embodiments, the texture of the composition and/or polymeric material may be such that it changes (e.g., increases, decreases) the wettability of the composition and/or polymeric material. Wettability may be determined, in some cases, by measuring the contact angle of a droplet of water with the surface of the polymeric material. In certain embodiments, the polymeric material may be textured such that at least a surface of the polymeric material is hydrophobic. In some embodiments, the contact angle of a droplet of water with the polymeric material comprising a textured surface may be at least about 80 degrees, at least about 90 degrees, at least about 95 degrees, at least about hundred degrees, at least about 110 degrees, or at least about 120 degrees.

In some embodiments, the compositions comprising a polymeric material and a therapeutic agent as described herein may increase the stability and/or the shelf life of the therapeutic agent as compared to traditional drug-delivery materials.

In another embodiment, the polymeric material is provided as a kit to an end-user. In some embodiments, the polymeric material is provided in a kit suitable for use with an additive manufacturing machine.

Any terms as used herein related to shape, orientation, alignment, and/or geometric relationship of or between, for example, one or more articles, compositions, structures, materials and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described.

The term “electrophile,” as used herein, refers to a functionality which is attracted to an electron and which participates in a chemical reaction by accepting an electron pair in order to bond to a nucleophile.

The term “nucleophile” as used herein, refers to a functionality which donates an electron pair to an electrophile in order to bond to a electrophile.

As used herein, the term “react” or “reacting” refers to the formation of a bond between two or more components to produce a stable, isolable compound. For example, a first component and a second component may react to form one reaction product comprising the first component and the second component joined by a covalent bond. The term “reacting” may also include the use of solvents, catalysts, bases, ligands, or other materials which may serve to promote the occurrence of the reaction between component(s). A “stable, isolable compound” refers to isolated reaction products and does not refer to unstable intermediates or transition states.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The alkyl groups may be optionally substituted, as described more fully below. Examples of alkyl groups include, but are not limited to, methyl, ethyl, propyl, isopropyl, butyl, isobutyl, tert-butyl, 2-ethylhexyl, cyclopropyl, cyclobutyl, cyclopentyl, cyclohexyl, and the like. “Heteroalkyl” groups are alkyl groups wherein at least one atom is a heteroatom (e.g., oxygen, sulfur, nitrogen, phosphorus, etc.), with the remainder of the atoms being carbon atoms. Examples of heteroalkyl groups include, but are not limited to, alkoxy, poly(ethylene glycol)-, alkyl-substituted amino, tetrahydrofuranyl, piperidinyl, morpholinyl, etc.

The terms “alkenyl” and “alkynyl” refer to unsaturated aliphatic groups analogous to the alkyl groups described above, but containing at least one double or triple bond respectively. The “heteroalkenyl” and “heteroalkynyl” refer to alkenyl and alkynyl groups as described herein in which one or more atoms is a heteroatom (e.g., oxygen, nitrogen, sulfur, and the like).

The term “aryl” refers to an aromatic carbocyclic group having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl), all optionally substituted. “Heteroaryl” groups are aryl groups wherein at least one ring atom in the aromatic ring is a heteroatom, with the remainder of the ring atoms being carbon atoms. Examples of heteroaryl groups include furanyl, thienyl, pyridyl, pyrrolyl, N lower alkyl pyrrolyl, pyridyl N oxide, pyrimidyl, pyrazinyl, imidazolyl, indolyl and the like, all optionally substituted.

The terms “amine” and “amino” refer to both unsubstituted and substituted amines, e.g., a moiety that can be represented by the general formula: N(R′)(R″)(R′″) wherein R′, R″, and R′″ each independently represent a group permitted by the rules of valence.

The terms “acyl,” “carboxyl group,” or “carbonyl group” are recognized in the art and can include such moieties as can be represented by the general formula:

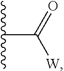

wherein W is H, OH, O-alkyl, O-alkenyl, or a salt thereof. Where W is O-alkyl, the formula represents an “ester.” Where W is OH, the formula represents a “carboxylic acid.” In general, where the oxygen atom of the above formula is replaced by sulfur, the formula represents a “thiolcarbonyl” group. Where W is a S-alkyl, the formula represents a “thiolester.” Where W is SH, the formula represents a “thiolcarboxylic acid.” On the other hand, where W is alkyl, the above formula represents a “ketone” group. Where W is hydrogen, the above formula represents an “aldehyde” group.

As used herein, the term “heterocycle” or “heterocyclyl” refers to a monocyclic or polycyclic heterocyclic ring that is either a saturated ring or an unsaturated non-aromatic ring. Typically, the heterocycle may include 3-membered to 14-membered rings. In some cases, 3-membered heterocycle can contain up to 3 heteroatoms, and a 4- to 14-membered heterocycle can contain from 1 to about 8 heteroatoms. Each heteroatom can be independently selected from nitrogen, which can be quaternized; oxygen; and sulfur, including sulfoxide and sulfone. The terms “heterocycle” or “heterocyclyl” may include heteroaromatic or heteroaryl groups, as described more fully below. The heterocycle may be attached via any heteroatom ring atom or carbon ring atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, the hydrogen on a nitrogen may be substituted with a tert-butoxycarbonyl group. Furthermore, the heterocyclyl may be optionally substituted with one or more substituents (including without limitation a halogen atom, an alkyl radical, or aryl radical).

As used herein, the term “heteroaromatic” or “heteroaryl” means a monocyclic or polycyclic heteroaromatic ring (or radical thereof) comprising carbon atom ring members and one or more heteroatom ring members (such as, for example, oxygen, sulfur or nitrogen). Typically, the heteroaromatic ring has from 5 to about 14 ring members in which at least 1 ring member is a heteroatom selected from oxygen, sulfur, and nitrogen. In another embodiment, the heteroaromatic ring is a 5 or 6 membered ring and may contain from 1 to about 4 heteroatoms. In another embodiment, the heteroaromatic ring system has a 7 to 14 ring members and may contain from 1 to about 7 heteroatoms. Representative heteroaryls include pyridyl, furyl, thienyl, pyrrolyl, oxazolyl, imidazolyl, indolizinyl, thiazolyl, isoxazolyl, pyrazolyl, isothiazolyl, pyridazinyl, pyrimidinyl, pyrazinyl, triazinyl, triazolyl, pyridinyl, thiadiazolyl, pyrazinyl, quinolyl, isoquinolyl, indazolyl, benzoxazolyl, benzofuryl, benzothiazolyl, indolizinyl, imidazopyridinyl, isothiazolyl, tetrazolyl, benzimidazolyl, benzoxazolyl, benzothiazolyl, benzothiadiazolyl, benzoxadiazolyl, carbazolyl, indolyl, tetrahydroindolyl, azaindolyl, imidazopyridyl, qunizaolinyl, purinyl, pyrrolo[2,3]pyrimidyl, pyrazolo[3,4]pyrimidyl, benzo(b)thienyl, and the like. These heteroaryl groups may be optionally substituted with one or more substituents.

The term “substituted” is contemplated to include all permissible substituents of organic compounds, “permissible” being in the context of the chemical rules of valence known to those of ordinary skill in the art. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl” must still comprise the phenyl moiety and cannot be modified by substitution, in this definition, to become, e.g., a heteroaryl group such as pyridine. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

Examples of substituents include, but are not limited to, alkyl, aryl, aralkyl, cyclic alkyl, heterocycloalkyl, hydroxy, alkoxy, aryloxy, perhaloalkoxy, aralkoxy, heteroaryl, heteroaryloxy, heteroarylalkyl, heteroaralkoxy, azido, amino, halogen, alkylthio, oxo, acyl, acylalkyl, carboxy esters, carboxyl, carboxamido, nitro, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, alkoxyalkyl, perhaloalkyl, arylalkyloxyalkyl, and the like.

As used herein, the term “network” refers to a three dimensional substance having oligomeric or polymeric strands interconnected to one another by crosslinks.

As used herein, the term “strand” refers to an oligomeric or polymeric chain of one monomer unit, or an oligomeric or polymeric chain of two or more different monomer units.

As used herein, the term “backbone” refers to the atoms and bonds through which the monomer units are bound together. As used herein, the term “pendent group,” when used in the context of the strand, refers to functional groups which are attached to the strand but do not participate in the bonds through which the monomer units are joined.

As used herein, the term “prepolymer” refers to oligomeric or polymeric strands which have not undergone crosslinking to form a network.

As used herein, the term “dynamic equilibrium” refers the process in which a network material rearranges its underlying chemical bonds. The rearrangement is characterized by the destruction and formation of individual chemical bonds throughout the network. The bonds involved in dynamic processes may be contained within the strand backbone, the pendent groups, or both.

As used herein, the term “observable dynamic equilibrium” refers to instances when the dynamic equilibrium rate is sufficiently high for the network material to be reformable. One of ordinary skill will appreciate that dynamic equilibrium processes take place to some extent at any temperature, but when the dynamic equilibrium rate is low, the network exhibits the characteristics of a thermoset.

As used herein, the term “crosslink” refers to a connection between two strands. The crosslink may either be a chemical bond, a single atom, or multiple atoms. The crosslink may be formed by reaction of a pendant group in one strand with the backbone of a different strand, or by reaction of one pendant group with another pendant group. Crosslinks may exist between separate strand molecules, and may also exist between different points of the same strand.

As used herein, the term “active substance” refers to a compound or mixture of compounds which causes a change in a biological substrate. Exemplary classes of active substances in the medical and biological arts include therapeutic, prophylactic and diagnostic agents. The active substance may be a small molecule drug, a vitamin, a nutrient, a biologic drug, a vaccine, a protein, an antibody or other biological macromolecule. The active substance may also be a fertilizer, a pesticide, an insecticide, an insect repellant, a herbicide or other biological active agent. The active substance may be a mixture of any of the above listed types of compounds.

“Immunosuppressive drug” refers to a drug that inhibits or prevents an immune response to a foreign material in a subject. Immunosuppressive drug generally act by inhibiting T-cell activation, disrupting proliferation, or suppressing inflammation.

As used herein, the terms “oligomer” and “polymers” each refer to a compound of a repeating monomeric subunit. Generally speaking, an “oligomer” contains fewer monomeric units than a “polymer.” Those of skill in the art will appreciate that whether a particular compound is designated an oligomer or polymer is dependent on both the identity of the compound and the context in which it is used.

One of ordinary skill will appreciate that many oligomeric and polymeric compounds are composed of a plurality of compounds having differing numbers of monomers. Such mixtures are often designated by the average molecular weight of the oligomeric or polymeric compounds in the mixture. As used herein, the use of the singular “compound” in reference to an oligomeric or polymeric compound includes such mixtures.

As used herein, reference to any oligomeric or polymeric material without further modifiers includes said oligomeric or polymeric material having any average molecular weight. For instance, the terms “polyethylene glycol” and “polypropylene glycol,” when used without further modifiers, includes polyethylene glycols and polypropylene glycols of any average molecular weight.

As used herein, the term “Michael acceptor” refers to a functional group having a carbon-carbon double or triple bond in which at least one of the carbon atoms is further bonded to a carbonyl group or carbonyl analogs such as imine, oxime, and thiocarbonyl. The reaction between a Michael acceptor and nucleophile results in the formation of a covalent bond between the nucleophile and the carbon atom not directly connected to the carbonyl group or carbonyl analog. The reaction between a Michael acceptor and a nucleophile may be called a “Michael addition.”

The term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

The term “alkoxy” refers to an alkyl group, as defined above, having an oxygen atom attached thereto. Representative alkoxy groups include methoxy, ethoxy, propyloxy, and tert-butoxy. An “ether” is two hydrocarbons covalently linked by an oxygen.

The term “alkylthio” refers to an alkyl group, as defined above, having a sulfur atom attached thereto. In some embodiments, the “alkylthio” moiety is represented by one of —S— alkyl, —S-alkenyl, and —S-alkynyl. Representative alkylthio groups include methylthio and ethylthio.

The term “amido” is art-recognized as an amino substituted by a carbonyl group.

The term “aralkyl”, as used herein, refers to an alkyl group substituted with an aryl group. The term “heteroaralkyl”, as used herein, refers to an alkyl group substituted with a heteroaryl group.

The term “heteroatom” as used herein means an atom of any element other than carbon or hydrogen. Examplary heteroatoms are nitrogen, oxygen, and sulfur.

As used herein, the term “thiol” means —SH; the term “hydroxyl” means —OH; and the term “sulfonyl” means —SO₂—.

As used herein the term “oxo” refers to a carbonyl oxygen atom.

As used herein, the term “alkaloid” refers to a naturally occurring organic compound containing at least one non-peptidic nitrogen atom.

As used herein, the term “leaving group” refers to a chemical moiety which is displaced during a substitution or elimination reaction. By way of example, the following moieties can function as leaving groups: chlorine, bromine, iodine, fluorine, and alkoxysulfonyl, and aryloxysulfonyl groups such as mesylate, trifluoromesylate, tosylate, besylate, and nosylate.

“Microparticle”, as used herein, generally refers to a particle having a diameter, such as an average diameter, from about 1 micron to about 100 microns, about 1 to about 50 microns, about 1 to about 30 microns, or about 1 micron to about 10 microns. The microparticles can have any shape. Microparticles having a spherical shape are generally referred to as “microspheres”.

“Nanoparticle,” as used herein, generally refers to a particle of any shape having an average diameter from about 1 nm up to, but not including, about 1 micron, about 5 nm to about 500 nm, or about 5 nm to about 300 nm. In some embodiments, the particles have an average diameter from about 100 nm to about 300 nm, about 100 nm to about 250 nm, or about 100 nm to about 200 nm. Nanoparticles having a spherical shape are generally referred to as “nanospheres”.

“Mean particle size,” as used herein, generally refers to the statistical mean particle size (diameter) of the particles in a population of particles. The diameter of an essentially spherical particle may be referred to as the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution,” are used interchangeably herein and describe a plurality of liposomal nanoparticles or microparticles where the particles have the same or nearly the same diameter or aerodynamic diameter. As used herein, a monodisperse distribution refers to particle distributions in which 75, 76, 77, 78, 79, 80, 81, 82, 83, 84, 85, 86, 86, 88, 89, 90, 91, 92, 93, 94, 95% or greater of the distribution lies within 85, 80, 75, 70, 65, 60, 55, 50, 45, 40, 35, diameter or aerodynamic diameter.

### Examples

The following examples are intended to illustrate certain embodiments of the present invention, but do not exemplify the full scope of the invention.

### Example 1

### Synthesis of Polymeric Materials Containing PEG and Citric Acid

76 mg of caffeine (0.1 mol, 5 mol %) and 823 mg of citric acid (1.0 mol) were weighed into a 20 mL vial equipped with a magnetic stirbar. 2 mL of diglycidyl PEG was added via syringe. A cap was placed on the vial and the mixture was stirred at 70° C. for five minutes. If any visual particles remain after this time, they may be crushed using a spatula to create a homogenous solution. The mixture was stirred until the viscosity of the mixture increased so as to indicate approach of the gel point. The mixture was poured into a silicon mold and placed in an oven set at 90° C.

As demonstrated by the table below, increasing bake durations were associated with increased tensile strength.

| Example | Time (hours) | Tensile strength (N/mm²) |
|---|---|---|
| A | 12 | 0.06 ± 0.008 |
| B | 24 | 0.164 ± 0.027 |
| C | 168 | 2.017 ± 0.37 |

Gel Formation Monitored by ¹H NMR and FT-IR:

¹H NMR spectra were collected during gel formation on a Bruker 300 in DMSO-d⁶. Samples were taken from the active polymerization using a glass pipette to swab the mixture and collect a representative sample. The swab was dissolved using DMSO-d⁶ into the NMR tube and frozen until data was collected.

FT-IR spectra were collected using a Bruker Alpha FT-IR. Samples of polymer were used to cover the detection window and measurements were taken directly of the polymerization without other preparation.

As shown in FIGS. 1H and 1J, the stacked FT-IR and ¹H NMR spectra show gel formation via the appearance of the signature ester peak at 1755 cm⁻¹ (denoted by #) and the disappearance of the carboxylic acid peak at 12.5 ppm (denoted by *), respectively from time (t)=0 to 600 minutes of the reaction.

### Example 2

### Evaluation of Absorption Properties of Polymeric Materials

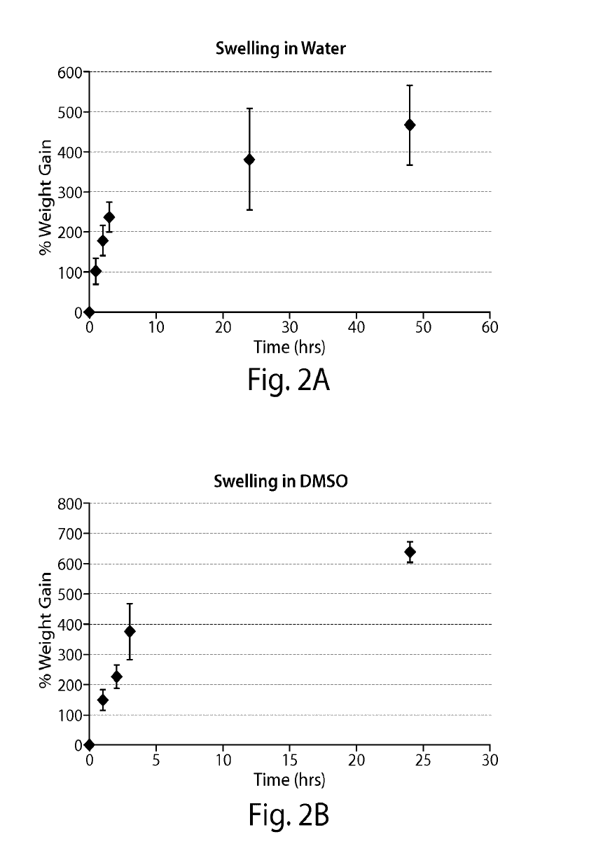

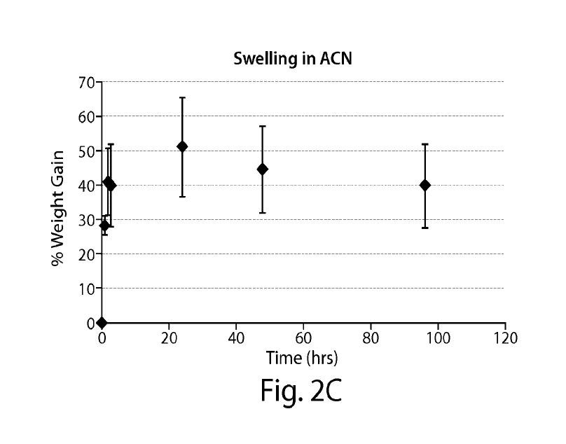

Samples of Example 1B were submerged in water, DMSO or acetonitrile at room temperature. FIGS. 2A-C depicts the weight gain of the polymeric material as a function of time at 5% catalyst.

To demonstrate the effect of the catalyst loading on hydrogel properties, the process of Example 1B was repeated, using catalyst loadings of 5%, 10%, 15%, 20% and 25%. FIGS. 3A-C depicts the weight gain of the network materials as a function of time.

### Example 3

### Synthesis of Polymeric Materials

The following polymeric materials were prepared according to the procedures described above. In each example, the baking conditions were 24 hours at 90° C. As shown in the examples herein, polymeric materials of varying physical properties (e.g., mechanical strength, hardness) can be obtained through the selection of different monomer units.

| Reagents | Molar Ratio | Y.M. | T.S. | Elong | X-link |
|---|---|---|---|---|---|
| PEG:caf:CA | 1:0.05:1 | 0.305 | 0.164 | 61.12 | |
| PEG:caf:CA | 1:0.1:1 | 0.300 | 0.139 | 53.31 | 24.79 |
| PEG:caf:CA | 1:0.15:1 | 0.479 | 0.197 | 45.86 | 15.52 |
| PEG:caf:CA | 1:0.20:1 | 0.418 | 0.216 | 67.28 | 17.78 |
| PEG:caf:CA | 1:0.25:1 | 0.507 | 0.181 | 40.81 | 14.66 |
| PEG:caf:CA:pluronic 407 | 1:0.1:1:0.02 | 0.690 | 0.567 | 89.04 | 10.77 |
| PEG:caf:CA:adipic acid | 1:0.1:0.5:0.5 | 0.690 | 0.347 | 61.52 | 10.78 |
| PEG:caf:CA:adipic acid | 1:0.1:0.75:0.25 | 0.723 | 0.286 | 44.02 | 10.29 |
| PEG:caf:CA:thiodipropionic acid | 1:0.1:0.5:0.5 | 0.196 | 0.167 | 111.87 | 37.90 |
| PEG:caf:CA:thiodipropionic acid | 1:0.1:1:0.5 | 0.973 | 0.262 | 29.32 | 7.64 |
| PEG:caf:CA:γ-decalactone | 1:0.1:1:0.5 | 0.222 | 0.195 | 97.48 | 33.44 |
| PEG:caf:CA:γ-decalactone | 1:0.1:0.5:0.5 | 0.333 | 0.203 | 70.79 | 22.32 |
| PEG:caf:CA:δ-decalactone | 1:0.1:0.5:0.5 | 0.474 | 0.367 | 78.00 | 15.70 |
| PEG:Zn(sulfate):fumaric | 1:0.1:0.5:0.5 | 0.214 | 0.117 | 107.38 | 34.67 |
| acid:δ-decalactone | | | | | |
| PPO3:caf:CA:PEG | 1:0.1:2:1 | 0.267 | 0.371 | 155.53 | 27.83 |
| PPO3:caf:CA | 1:0.1:1 | 4.77 | 0.833 | 26.78 | 1.56 |
| PPO3:caf:CA:triethyl citrate | 1:0.1:1:0.2 | 1.765 | 0.365 | 27.98 | 4.21 |
| PPO3:caf:CA:propylene glycol | 1:0.1:1:0.2 | 0.768 | 0.266 | 65.70 | 9.67 |
| PPO3:caf:CA:sebacic acid | 1:0.1:0.5:0.5 | 0.919 | 0.649 | 83.26 | 8.09 |
| PPO3:caf:CA:adipic acid | 1:0.1:0.5:0.5 | 1.038 | 0.395 | 43.32 | 7.16 |
| PPO3:caf:CA:succinic acid | 1:0.1:0.5:0.5 | 0.799 | 0.602 | 88.86 | 9.30 |
| PPO3:caf:CA:fumaric acid | 1:0.1:0.5:0.5 | 1.200 | 0.503 | 48.16 | 6.19 |
| PPO3:caf:CA:δ-decalactone | 1:0.1:0.5:0.5 | 1.367 | 0.545 | 27.36 | 5.44 |
| PPO3:caf:CA:γ-decalactone | 1:0.1:0.5:0.5 | 0.968 | 0.476 | 57.34 | 7.68 |
| PPO3:caf:CA:γ-decalactone | 1:0.1:0.5:0.5 | 1.033 | 0.327 | 31.26 | 7.19 |
| PPO3:caf:CA:γ-decalactone | 1:0.1:0.5:0.5 | 1.226 | 0.559 | 49.84 | 6.06 |
| PPO3:caf:CA:ω-pentadecalactone | 1:0.1:1:0.5 | 3.211 | 0.230 | 30.96 | 2.31 |
| PPO3:caf:CA:ω-pentadecalactone | 1:0.1:0.5:0.5 | | 0.141 | 59.58 | |
| PPO3:caf:CA:caprolactam | 1:0.1:0.5:0.5 | 0.521 | 0.188 | 40.48 | 14.26 |
| PPO3:caf:CA:PPO6 | 1:0.1:1:1 | 0.217 | 0.115 | 59.44 | 34.25 |
| PPO3:caf:CA:g-decalactone:PPO6 | 1:0.1:0.5:0.5:1 | 0.770 | 0.295 | 41.79 | 9.66 |
| PPO3:caf:CA:]γ-decalactone:PPO6 | 1:0.1:0.75:0.25:1 | 0.546 | 0.352 | 72.68 | 13.62 |
| PPO3:caf:CA:caprolactam:PPO6 | 1:0.1:0.5:0.5:1 | 0.367 | 0.177 | 53.61 | 20.24 |
| PPO3:caf:CA:dithiodipropionic | 1:0.1:0.75:0.25:1 | 0.113 | 0.141 | 141.53 | 65.99 |
| acid:PPO6 | | | | | |
| PPO3:caf:CA:δ- | 1:0.1:0.5:0.5:1:0.2 | 0.610 | 0.272 | 49.10 | 12.19 |
| decalactone:PPO6:stearic acid | | | | | |
| PPO3:caf:CA:adipic acid:δ- | 1:0.1:0.75:0.25:0.25:1:0.2 | 0.521 | 0.218 | 46.16 | 14.26 |
| decalactone:PPO6:Propylene | | | | | |
| Glycol | | | | | |
| PPO6:caf:γ- | 1:0.1:0.5:1:1 | 1.538 | 0.430 | 28.88 | 4.83 |
| decalactone:CA:trimethylol | | | | | |
| propane glycidyl ether | | | | | |
| PPO6:caf:dithiopropionic | 1:0.1:1:1:1 | 1.314 | 0.415 | 33.66 | 5.66 |
| acid:CA:trimethylol propane | | | | | |
| glycidyl ether | | | | | |
| PPO3:caf:CA:mercaptosuccinic | 1:0.1:0.5:0.5:0.25 | 0.31 | 0.27 | 96.85 | 23.62 |
| acid:PPO dimethacrylate | | | | | |
| PPO3:caf:CA:mercaptosuccinic | 1:0.1:0.5:0.5:0.25 | 0.79 | 0.45 | 62.34 | 9.45 |
| acid:PPO6 | | | | | |
| PPO3:caf:CA:fumaric | 1:0.1:0.5:0.5:0.25 | 1.27 | 0.62 | 55.35 | 5.85 |
| acid:methylenebis(acrylamide) | | | | | |
| PEG:caf:CA:WC (weight percent) | 1:0.1:1:0.01 | 0.24 | 0.17 | 77.59 | 31.50 |
| PEG:caf:CA:WC (weight percent) | 1:0.1:1:0.05 | 0.24 | 0.16 | 82.66 | 30.59 |
| PEG:caf:CA:WC (weight percent) | 1:0.1:1:0.1 | 0.14 | 0.11 | 129.58 | 53.09 |
| 1,4-butanediol:caf:CA | 1:0.1:1 | 135.94 | 4.411 | 13.09 | 0.05 |
| neopentyl ether:caf:CA | 1:0.1:1 | 37.408 | 1.272 | 38.80 | 0.20 |
| PEG:Zn(sulfate):fumaric acid | 1:0.1:1 | 0.47 | 0.15 | 34.67 | 15.81 |
| PEG:Zn(sulfate):fumaric | 1:0.1:1:0.1 | 0.27 | 0.13 | 55.68 | 27.10 |
| acid:trehalose | | | | | |
| PPO3:Zn(sulfate):fumaric | 1:0.1:0.5:0.5 | 0.21 | 0.11 | 55.40 | 34.73 |
| acid:decalactone | | | | | |

Key -
Y.M. = Young's Modulus (N/mm²)
T.S. = Tensile Strength (N/mm²)
elong = Elongation at break
X-link = Crosslinking density (mol/m³)
PEG = polyethylene glycol, diglycidyl ether, Mn = 500 g/mol
PPO3 = polypropylene glycol, diglycidyl ether, Mn = 380 g/mol
PPO6 = polypropylene glycol, diglycidyl ether, Mn = 640 g/mol
PPO dimethacrylate = polypropylene glycol dimethacrylate, Mn = 580 g/mol
Caf = caffeine
CA = citric acid
PLU = pluronic 26 kDa
WC = tungsten carbide, ratio unit is expressed in weight percent.

Adventitious Water not a Majority Component of Polymeric Materials Synthesized:

Small discs (2 mm H×10 mm W) of materials listed in table below were placed in a vacuum (23 mmHg) oven at 60° C. and allowed to dry for 24 hours. Plain drying under vacuum leads to some mass loss reflective of adventitious water that was included from the atmosphere and/or during synthesis.

| Material | % Mass Loss on Drying |
|---|---|
| PEG, caf, CA (5 mol % caf) | −3 |
| PPO, caf, CA (5 mol % caf) | −2 |
| PEG-PPO, caf, CA (5 mol % caf) | −2 |
| PEG, CA (no catalyst) | −4 |
| PPO, CA (no catalyst) | −2 |

Mass Loss in Polymeric Materials Synthesized During Swelling Compares to Catalyst Loading:

Small discs (2 mm H×10 mm W) of materials listed in table below were swelled for 24 hours in a good solvent (ethyl acetate) to demonstrate that the material does not dissolve. The discs were subsequently dried under vacuum (23 mmHg) with gentle heating (60° C.) to remove solvent.

| Material | % Swelling | % Loss |
|---|---|---|
| PEG, caf, CA (5 mol % caf) | 23 | −10 |
| PPO, caf, CA (5 mol % caf) | 38 | −9 |
| PEG-PPO, caf, CA (5 mol % caf) | 23 | −10 |
| PEG, CA (no catalyst) | 18 | −31 |
| PPO, CA (no catalyst) | 28 | −28 |

The mass loss via swelling/leaching is presumed to represent unbound chemical species. This correlates with the mass of catalyst used in the first three systems, where the percent mass loss after leaching organics into ethyl acetate was about 10% for structures synthesized using 10% catalyst loading. For materials synthesized without the use of a catalyst the mass loss was much higher suggesting an incomplete reaction and significant loss of monomers.

Material Composition Impacts Response to Solvent as Measured by Solvent Absorption:

Small discs (2 mm H×10 mm W) of PEG, PEG-PPO, and PPO materials with citric acid (CA) were incubated in simulated biologics (SGF, SIF, PBS) and organic solvents (ethanol, ethyl acetate, and hexanes) at room temperature for 24 hours. After 24 hours, the materials were removed from the respective solvent, dried lightly with a Kim Wipe and massed.

As shown in FIG. 4, the wetting dynamics of three chemically different materials reflects the internal environment of the materials as shown in the representative response to incubation in the simulated biologics and organic solvents as measured by the percent mass change.

Hydration Kinetics Impacted by Material Composition as Measured by Solvent Absorption:

Small discs (2 mm H×10 mm W) of materials were incubated in simulated biologics (SGF, SIF, PBS) at 37 C for different amounts of time (0-5 hours) and subsequently removed at a particular time point and massed. The samples were briefly dried by dabbing with a Kim Wipe to remove any residual solvent.

FIG. 5 shows the hydration kinetics of the materials in the simulated biological solvents as measured by the percent mass change.

### Example 4

### Moldability of Polymeric Materials Containing Artesunate

Networks were prepared using the process and ingredients in Example 1 with the addition of artesunate. The rings were prepared as described above in Example 1. Each of the rings was loaded with 480 mg of artesunate.

A comparison of the mechanical properties of loaded and unloaded network is shown in the table below:

| | | | | | Cross- |
|---|---|---|---|---|---|
| | | Tensile | % Elon- | Young's | linking |
| | | Strength | gation | Modulus | Density |
| PEG:caf:CA | Average | 0.14 | 53.31 | 0.30 | 38.07 |
| 1:0.1:1 | SD | 0.03 | 5.53 | 0.03 | 4.01 |
| PEG:caf:CA:Artes- | Average | 0.27 | 42.89 | 0.95 | N/A |
| unate | | | | | |
| 1:0.1:0.5:0.64 | SD | 0.11 | 16.96 | 0.17 | N/A |

The introduction of an active substance into the network material does not compromise the mechanical integrity of the polymeric material nor the ability to form complex shapes using the polymeric material. In fact, upon loading with drug, the strength of the network increased and does not lose its ability to elongate. The loaded network does exhibit an increased Young's modulus.

### Example 5

### Moldability of Polymeric Materials

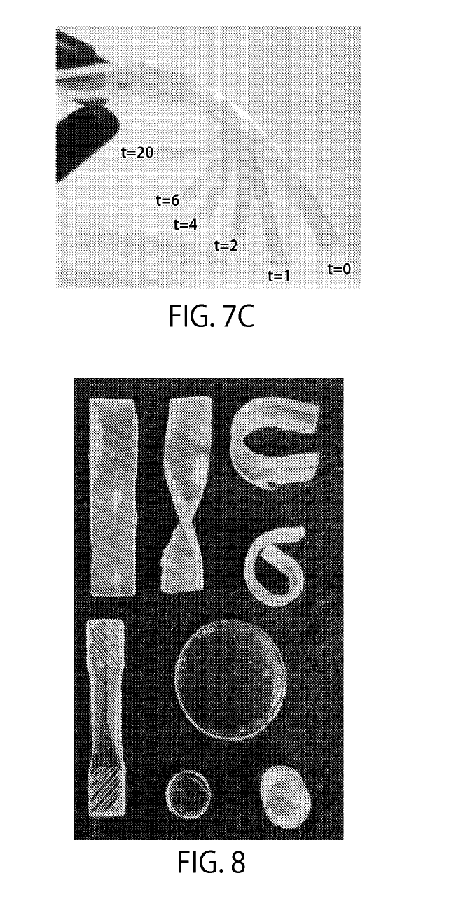

Articles made from the polymeric material may be manipulated in a variety of ways. Rectangular flat blocks, as described in Example 1, were prepared. These blocks were placed in a horseshoe-shaped fixture (FIGS. 7A-B) and incubated at 90° C. for varying time points (t=0-20 hours). The flat rectangular blocks were suspended parallel to the ground and photographed on edge (FIG. 7C)

In addition to bending, the network material may also be rolled, twisted, and/or bent (FIG. 8).

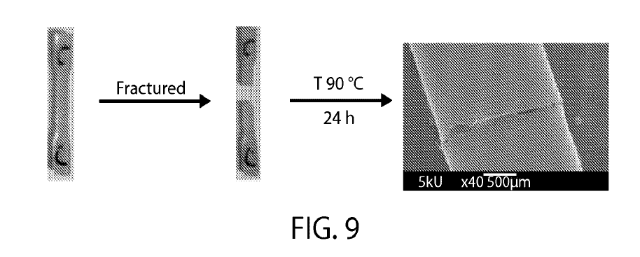

Separate articles made from the polymeric material may be joined together (e.g., rehealed) in the presence of heat. In one embodiment, a fractured article may be repaired as depicted in FIG. 9. In this example, a polymeric material formed by the reaction of PEG and citric acid (1:1 molar ratio with 5 mol % caffeine) showed a 3% loss in strength after breaking and rehealing as compared to the original material. A comparative polymeric material formed by the reaction of PEG, citric acid, and adipic acid (1:0.5:0.5 molar ratio with 5 mol % caffeine) had a 63% loss in strength.

In another embodiment, two separate articles may be joined together to construct an article which is not easily obtained from a single mold. The fusion between the separate articles has a mechanical strength similar to an individually molded article.

### Example 6

### Textured Polymeric Materials with Different Surface Properties

A lotus leaf was obtained from a farm (Florida), and affixed to a petri dish, covered in silicon and a negative of the lotus leaf was produced. Polymeric materials (PEG, PEG-PPO, and PPO) as described above were cast onto the negative lotus leaf mold.

The morphologies of the fabricated PEG, PEG-PPO, and PPO surfaces were examined by scanning electron microscopy (SEM; JEOL 5600LV, 5 kV, ×330). The samples were first sputter-coated with carbon using a Hummer 6.2 Sputter Coating System and then cut to be under 0.5 cm² in area and affixed onto an aluminum stub with double-sided adhesive carbon conductive tape.

The fabricated surfaces were further characterized for degree of adhesiveness and hydrophobicity by taking the static contact angle using a Kruss Drop Shape Analyzer DSA 100 (Drop Shape Analyzer software). Contact angles of water droplets over the PEG, PEG-PPO, and PPO fabricated surfaces were fixed to lay flat on a horizontal plane and the measurements were taken at room temperature. A fixed volume of ˜250 μL droplet was dispensed onto the substrate and then the contact angle made between the line tangent to the liquid droplet and the substrate surface was measured. The macroscopic droplet profile was photographed by a camera within the instrument. For each surface, eight contact angle measurements were taken. The average and the standard deviation values of each surface were calculated.

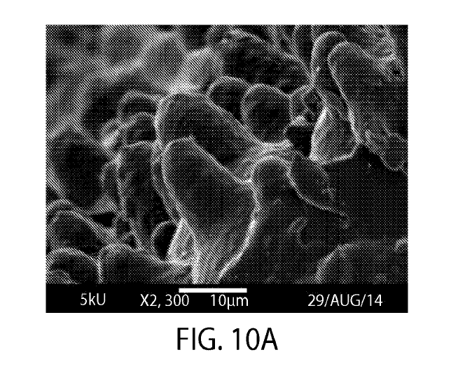

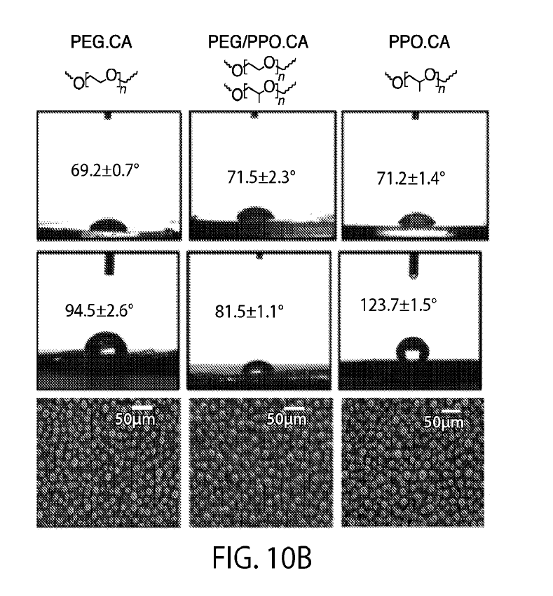

FIG. 10A shows a scanning electron microscopy image of an exemplary textured surface.

FIG. 10B shows static contact angles using dH₂O on the surface of silicon molded PEG, PEG-PPO, and PPO polymeric materials having no specific texturing and having lotus texturing on the surface. The bottom of FIG. 10B shows SEM images taken of the lotus leaf textured polymeric materials. The contact angles of the surfaces without specific texturing and having lotus leaf texturing were as follows:

| PEG | PEG-PPO | PPO |
|---|---|---|
| 69.2 ± 0.7° | 71.5 ± 2.3° | 71.2 ± 1.4° |
| 94.5 ± 2.6° | 81.5 ± 1.1° | 123.7 ± 1.5° |

### Example 7

### PPO, PEG-PPO, and PEG Characterization

Tensile Stress-Strain Curves:

Quasi-static test-to-failure (0.05 mm/s displacement rate) was conducted for n=10 samples. Standard “dogbone” shapes (modeled after ASTM D412; dimensions: 2 mm H×41 mm L×8 mm Wa×6 mm Wb) were cured by injecting viscous fluid into 3-D printed molds. The width, thickness, and gauge length of each sample was measured prior to testing using digital calipers.

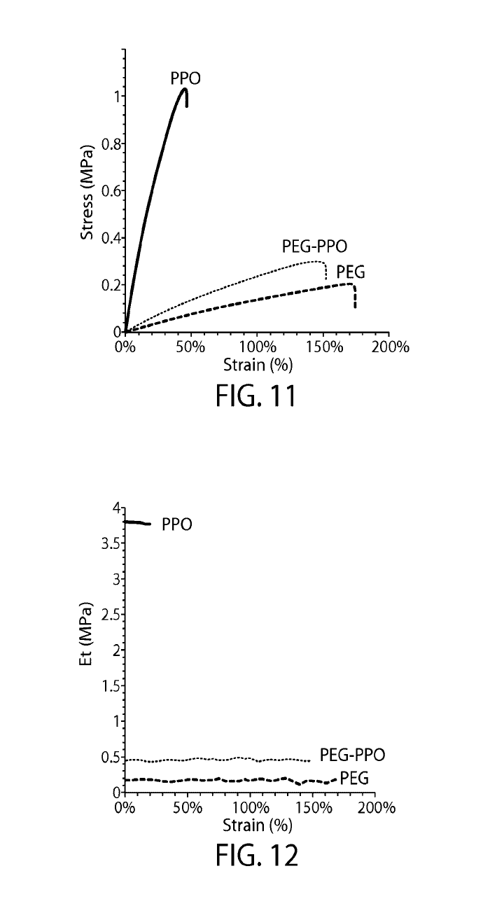

As shown in the tensile stress-strain curves for PPO, PEG-PPO, and PEG in FIG. 11, the elastic modulus and ultimate tensile strength of PPO is much larger that PEG-PPO and PEG. The extensibility of PEG-PPO and PEG, however, were much greater than for PPO.

Mean Elastic Modulus:

The elastic modulus of PPO, PEG-PPO, and PEG was calculated by differentiating the stress-strain curve using an automated MatLab. As shown in FIG. 12, PPO had the largest elastic modulus and PEG-PPO had a significantly larger mean elastic modulus value than PEG.

The elastic moduli of the materials showed that they can be tuned by varying the amount of PPO and PEG allowing for optimization of the mechanical properties of each based on intended use.

Compressive Strain-Stress Curves:

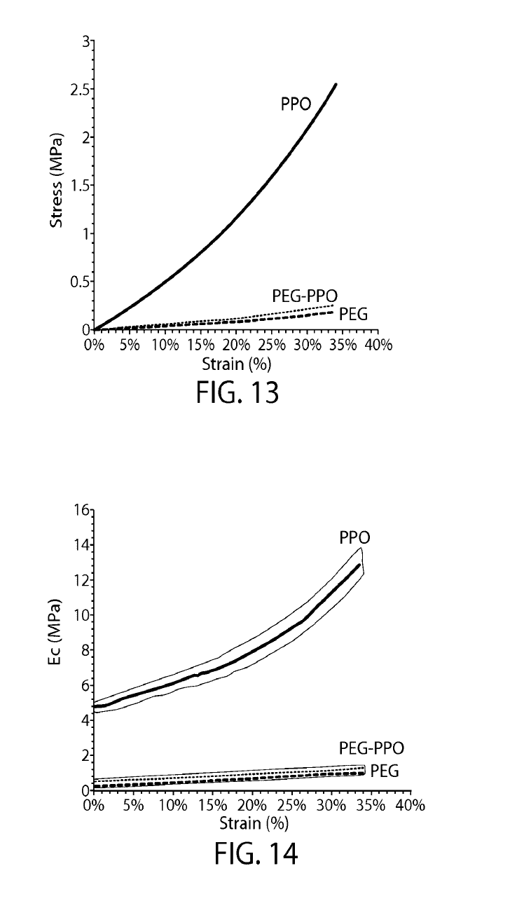

Compressive strain-stress curves for PPO, PEG-PPO, and PEG materials are shown in FIG. 13. Compressive tests (0.05 mm/s displacement rate) were conducted for n=10 samples. Standard disc shapes (2 mm H×25 mm W) were cured by injecting viscous fluid into 3-D printed molds. The diameter and height of each sample was measured prior to testing using digital calipers.

Mean Compressive Modulus:

Mean compressive moduli for PPO, PEG-PPO, and PEG materials are shown in FIG. 14. The compressive modulus was calculated by differentiating the stress-strain curve in an automated MatLab program. PPO was found have a mean compressive modulus which was significantly higher than those of the PEG-PPO and PEG materials which suggests that the compressive moduli can be tuned by varying the rate of PPO or PEG allowing for optimization of the mechanical properties of each depending on intended use.

Shear Stress—Shear Rate Curves:

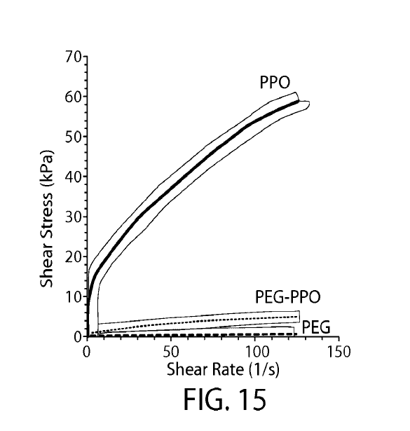

Shear stress—shear rate curves for PPO, PEG-PPO, and PEG materials are shown in FIG. 15. Shear tests (0.05 mm/s displacement rate) were conducted for n=10 samples. Standard disc shapes (2 mm H×25 mm W) were cured by injecting viscous fluid into 3-D printed molds. The diameter and height of each sample was measured prior to testing using digital calipers.

### Example 8

### Cytotoxicity

Polymers were dissolved in HCl and left at 70° C. overnight to dissolve completely. Subsequently the pH was adjusted to 7.0 using NaOH. The final polymer solution was diluted with Dulbecco's Modified Eagle Medium (DMEM) (Life Technologies) to 50 mg/mL before testing. Cytotoxicity was tested on HeLa, HEK293, C2BBe1 (ATCC) and HT29-MTX-E12 cells (Public Health England) by seeding them in a 96-well plate at a density of 6×10³, 16×10³, 16×10³ and 2×10⁴ cells/well respectively. HeLa and HEK293 cells were cultured in 100 μL DMEM containing 1% non-essential amino acids, 10% fetal bovine serum (FBS) and 1% penicillin-streptomycin solution (Life Technologies) per well. C2BBe1 and HT29-MTX-E12 cells were cultured in the same medium but was additionally supplemented with 4 mg/mL human transferrin (Life Technologies). Cells were kept in culture for 3 days before replacing the medium, to which the dissolved aqueous polymer solutions were added (final concentrations of polymers ranged from 0.078-20 mg/mL). After 72 h, cytotoxicity was quantified by adding 10 μL alamarBlue reagent (Life Technologies) to each well. The contents were mixed well and then allowed to incubate at 37° C. for 1 h. Absorbance at 570 nm was recorded on an Infinite M200Pro (Tecan) using 600 nm as reference wavelength. A positive control was provided by lysing cells with 1% Tween-20 and cells that were not subject to any polymer provided a negative control. Cell viability was calculated by the following equation: Cell viability (%)=100×(Absorbance₍ₛₐₘₚₗₑ₎−Absorbance_{(positive control)})/(Absorbance_{(negative control)}−Absorbance_{(positive control)}).

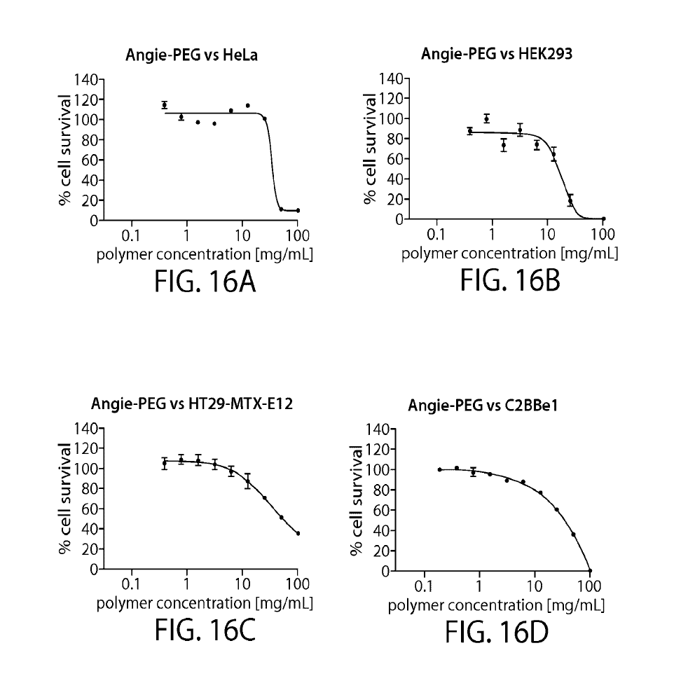

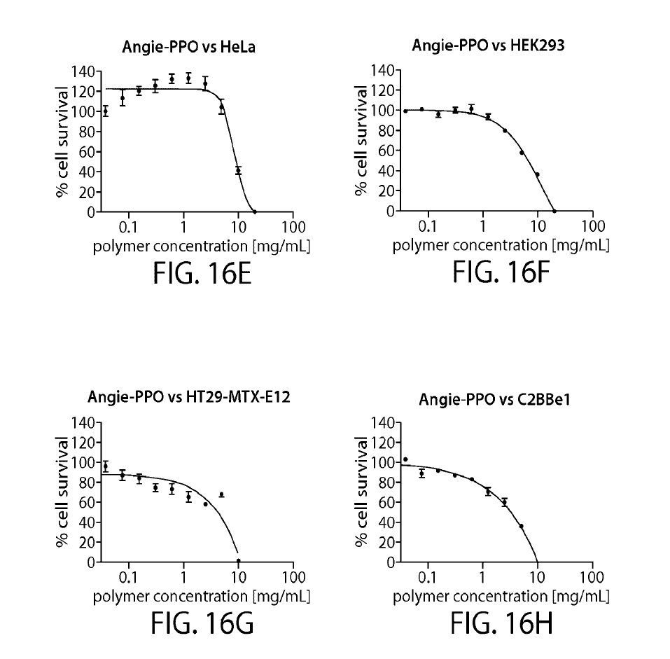

FIGS. 16A-16H show the percent cells surviving at various concentrations of polymer added to the medium.

Gels consisting of PPO:CA:delta-decalactone [1]: [0.5]: [0.5] were prepared in 30 mm glass dishes, sterilized using dry heat autoclave, and seeded at a density of 26,000 HeLa cells in 1.7 mL of high glucose pyruvate containing Dulbecco's Modified Eagle Medium (DMEMM) containing 1% non-essential amino acids, 10% fetal bovine serum (FBS) and 1% penicillin-streptomycin solution (Life Technologies). The samples were kept in culture for 3 days. After 3 days the cells were removed from the incubator, excess medium was removed, and a LIVE/DEAD assay (Life Technologies) was used to help visualize survival. The porosity of the gel precluded quantitative data, however, live cells adhered to the surface were easily visualized using standard light microscopy.

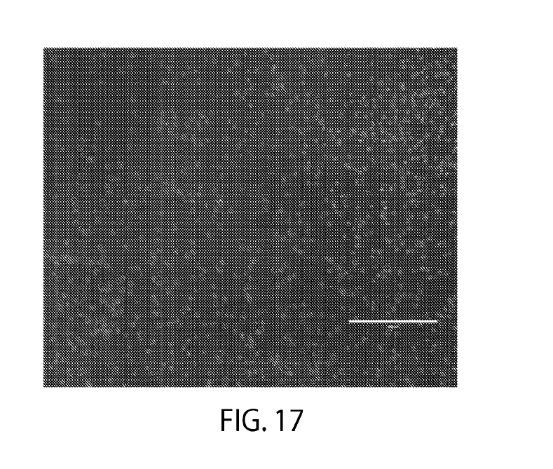

FIG. 17 is a an optical microscopy photograph of HeLa cells grown on a PPO:CA:delta-decalactone polymeric material.

### Example 9

### Drug Release

Small discs (2 mm H×10 mm W) were loaded with active pharmaceuticals (artesunate, dexamethasone, or ivermectin) at 20 weight % and processed as detailed above. The artesunate sample was incubated in 10 mL acetonitrile for 24 hours to remove all organic soluble compounds and isolate the drug. Leeched artesunate was compared to pure artesunate using HPLC to confirm structural integrity and stability through the manufacturing process. Ivermectin containing samples were incubated in 10 mL of simulated gastric fluid, incubated at 37° C. shaking at a speed of 500 rpm. Aliquots were removed for 5 time points and mass of drug released was quantified using HPLC.

HPLC:

The Agilent 1260 Infinity HPLC system equipped with Model 1260 quaternary pump, Model 1260 Hip ALS autosampler, Model 1290 thermostat, Model 1260 TCC control module, and Model 1260 diode array detector (DAD). The output signal was monitored and processed using the ChemStation® software. Analytical column was a 50-mm×4.6-mm EC-C18 Agilent Poroshell 120 column with 2.7-μm spherical particles. The mobile phase was filtered before use through a 20-μm Agilent nylon filter under reduced pressure.

Ivermectin Conditions:

50% ACN: 40% MeOH: 10% H2O—isocratic; 0.7 mL/min flow rate; 20 uL injection;

254 nm no ref.; room temperature; Sample in 100% MeOH; 10 min total runtime

Artesunate Conditions:

50% ACN: 50% H2O w/ 0.1% formic acid—isocratic; 0.9 mL/min flow rate;

20 uL injection; 210 nm with 360 nm ref.; room temperature; 10 min total runtime

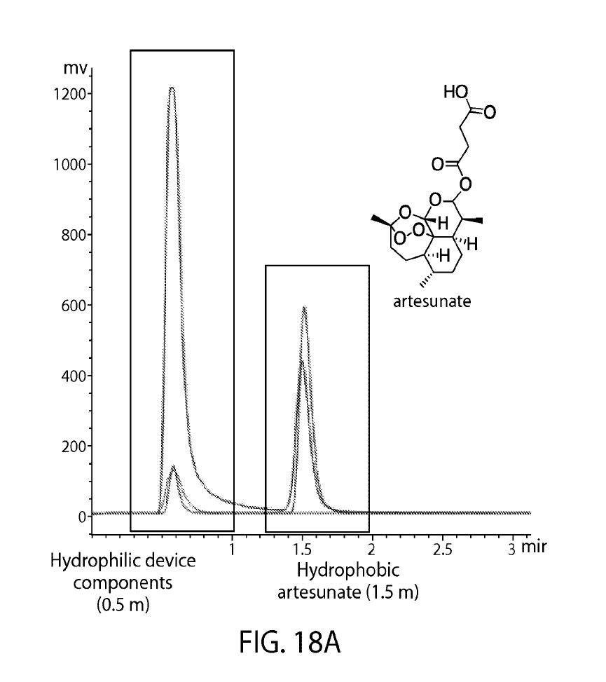

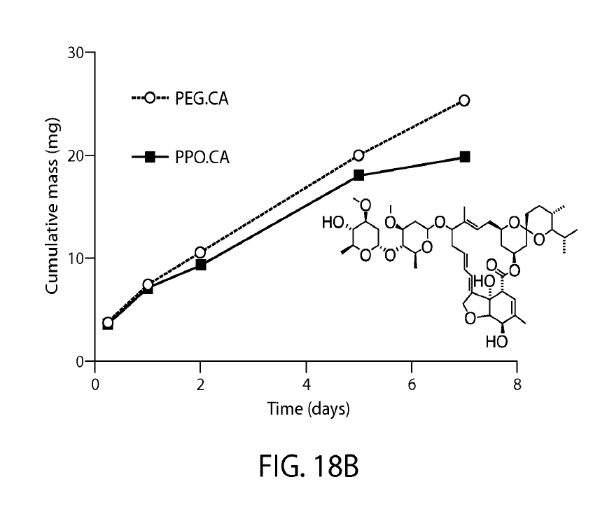

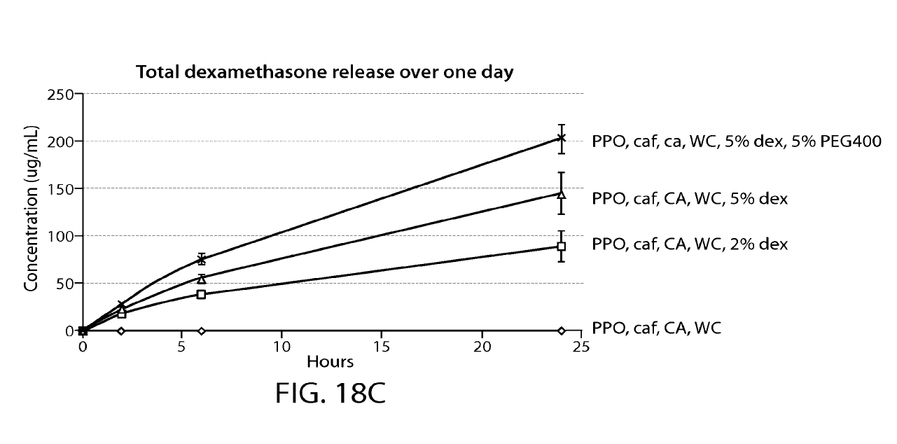

FIG. 18A shows the HPLC analysis of artesunate released from a PEG:CA polymeric material. FIG. 18B shows the cumulative mass of 20% ivermectin released from 0.2 g discs into simulated gastric fluid.

PPO:CA:caf 10 mm discs were also incubated in 5 mL of simulated gastric fluid and stirred at 37° C. for the time frame indicated. At each time point, the samples were removed from their solvent and placed into a fresh solution. The original solvent was then aliquoted and the mass of drug release assessed using HPLC.

Dexamethasone Conditions:

isocratic—30% acetonitrile, 70% water+0.1% formic acid pH3

254 nm, no ref. 1.0 mL/min flow rate. 10 min total runtime

20 uL injection volume room temperature ˜23 C retention time 3.2 min-3.6 min sample solvent—SIF w/ EtOH

FIG. 18C shows the concentration of dexamethasone released over 24 hours by various PPO:CA:caf based polymeric materials with increasing concentrations of dexamethasone.

### Example 10

### Mucoadhesion

| | | | Citric/ | Citric/ | Citric/ | |
|---|---|---|---|---|---|---|
| | Tartaric | Citric | dithiopropionic | adipic | sebacic | |
| | acid | acid | acid | acid | acid | Rod |
| Ratio(s): | 1:1.5 | 1:1 | 2:1:1 | 2:1:1 | 2:1:1 | — |
| Average | 0.85 | 0.50 | 0.51 | 0.57 | 0.54 | 0.01 |
| Force of | | | | | | |
| Adhesion | | | | | | |
| (N/cm²): | | | | | | |

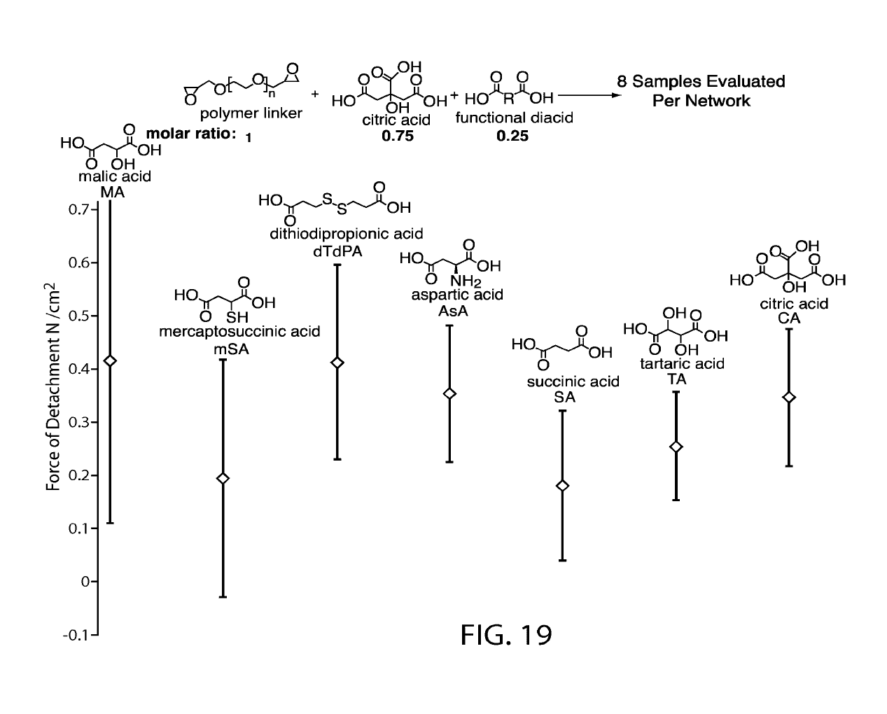

FIG. 19 shows a plot of ranges of forces of detachment in N/cm² for various functional diacids, listed above, added to a PEG:CA polymeric material during polymerization, with mucosal tissue. Testing was conducted at pH 1.1 and 6.7. The polymeric material was compressed into the tissue in an Instron for 5 minutes at a force of 0.5 N before the force required to detach the polymer was measured.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elipitical/elipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

What is claimed is:

1. A composition, comprising:
a crosslinked polymeric material and an active substance associated with the material;
wherein the crosslinked polymeric material comprises a polymer backbone and between 1 mol % and 25 mol % with respect to polymer agent a food grade catalyst, inclusive;
wherein the active substance is present in the composition in an amount of at least about 0.1 wt % based on the weight of the composition; and
wherein the composition comprises less than about 10 wt % auxiliary materials other than the crosslinked polymeric material, food grade catalyst, and the active substance, based on the weight of the composition.

2. A composition as in claim 1, wherein the composition is constructed and arranged to release the active substance from the crosslinked polymeric material.

3. A composition of any one of the preceding claims, wherein the composition releases between about 0.05 vol % and about 99.0 vol % of the active substance in less than about 196 hours.

4. A composition of any one of the preceding claims, wherein the composition contains at between about 0.01 wt % and about 40 wt % of the active substance versus the total composition weight.

5. A composition, comprising:
a crosslinked polymeric material formed by the reaction of one or more polyfunctional monomers and a food grade catalyst;
wherein the polymeric material comprises a bioresponsive bond; and
wherein the composition comprises less than about 10 wt % auxilliary materials other than the crosslinked polymeric material, the food grade catalyst, and, optionally, an active substance, based on the weight of the composition.

6. A composition, comprising:
a covalently crosslinked polymeric material formed by the reaction of a first polyfunctional monomer and a second polyfunctional monomer in the presence of a food grade catalyst;
wherein the first polyfunctional monomer comprises a first reactive group;
wherein the second polyfunctional monomer comprises a second reactive group capable of forming a covalent bond with the first reactive group;
wherein at least about 1% of the first reactive groups in the crosslinked polymeric material are free.

7. A composition as in claim 6, wherein the first and second polyfunctional monomers are the same or different and comprise the structure as in Formula (I):
Q¹-(CR¹R²)_{y}—X¹—X²—X³—(CR¹R²)_{z}-Q² (I)
wherein:
X¹, X², and X³ are the same or different and are absent or selected from the group consisting of (CR¹R²)ₘ, a heteroatom, an alkenyl, an alkynyl, a cycloalkyl, an aryl, a heterocyclic group, a heteroaryl group, an oligomeric group;
m, y and z are zero or any integer;
each R¹ and R² are the same or different and are selected from the group consisting of hydrogen, Q³, an aliphatic group, a halogen, a hydroxyl, a carbonyl, a thiocarbonyl, an oxo, an alkoxy, an epoxy, a phosphoryl, a phosphate, a phosphonate, a phosphinate, an amino, an amido, an amidine, an imine, a cyano, a nitro, an azido, a thiol, an alkylthio, a sulfate, a sulfonate, a sulfamoyl, a sulfonamido, a sulfonyl, a cycloalkyl, a heterocyclyl, an aralkyl, and an aromatic or heteroaromatic or a Michael acceptor, wherein any two or more Ra and Ra′ groups may be bonded together so as to form a ring system; and
Q¹, Q², and Q³ are the same or different and an electrophilic functional group or a nucleophilic functional group.

8. A composition as in claim 7, wherein the reaction of two of Q¹, Q², and Q³ forms an ester, ether, amide, thioether, amine bond.

9. A composition as in any one of claims 6-8, wherein the crosslinked polymeric material is formed by the reaction with an additional monomeric unit.

10. A composition, comprising:
a covalently crosslinked polymeric material, wherein the covalently crosslinked polymeric material comprises a bioresponsive bond and is formed via a reaction catalyzed by a food-grade catalyst;
wherein the covalently crosslinked polymeric material comprises at least about 1% free reactive groups; and
wherein the covalently crosslinked polymeric material is thermally reconfigurable.

11. A composition of any one of the preceding claims, wherein the composition comprises a an active substance associated with the covalently crosslinked polymeric material.

12. A composition as in claim 11, wherein the active substance is present in the composition in an amount of at least about 0.01 wt % based on the weight of the composition.

13. A composition of any one of the preceding claims, wherein the composition is constructed and arranged to release the active substance from the composition.

14. A composition of any one of the preceding claims, wherein the composition is constructed and arranged to release at least about 1 vol % of the active substance for a period of time of at least about 196 hours.

15. A composition of any one of the preceding claims, wherein the active substance is released via diffusion out of the crosslinked polymeric material.

16. A composition of any one of the preceding claims, wherein the active substance is released via degradation of the crosslinked polymeric material.

17. A composition of any one of the preceding claims, wherein the composition is biocompatible.

18. A composition of any one of the preceding claims, wherein the composition is biodegradable.

19. A composition of any one of the preceding claims, wherein the auxilliary materials comprise solvent, non-food grade catalysts, and/or excipients.

20. A composition of any one of the preceding claims, wherein the composition comprises substantially no solvent.

21. A composition of any one of the preceding claims, wherein the polymeric material comprises an ester bond.

22. A composition of any one of the preceding claims, wherein the food-grade catalyst is selected from the FDA's “Generally Recognized as Safe” Substances database and/or listed in 21 C.F.R. §182.

23. A composition of any one of the preceding claims, wherein the active substance is crystalline.

24. A composition of any one of the preceding claims, wherein the active substance is covalently bound to the crosslinked polymer network.

25. A composition of any one of the preceding claims, wherein the active substance has an average largest cross-sectional dimension greater than the average pore size of the crosslinked polymeric material.

26. A composition of any one of the preceding claims, wherein the free reactive group comprises carboxylic acid.

27. A dynamic network comprising a cross-linked prepolymer, wherein the dynamic network exhibits an observable dynamic equilibrium at a temperature greater than 40° C.

28. The dynamic network of claim E1, further characterized by at least one of the following:
a cross linking density between 65-550 mol/m³,
a Young's modulus between 0.01-500 N/mm²;
a tensile strength of 0.1-5 N/mm²;
the ability to absorb water or solvent in an amount of at least 10% of the dry weight of the network material.

29. A dynamic network comprising a cross-linked polymer, wherein the polymer has a structure of Formula (1): 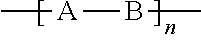
wherein A and B are derived from one or more monomers of Formula (2) and (3),
wherein the monomer of Formula (2) has the structure: 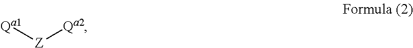
wherein Q^{a1} and Q^{a2} are electrophilic functional groups and Z has a structure selected from: 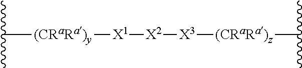
wherein [symbol] indicates a point of connection of Z to either Q^{a1} or Q^{a2},
wherein y and z are in each case independently selected from zero or any integer,
wherein R^{a} and R^{a′} are in each case independently selected from hydrogen, an aliphatic group, a halogen, a hydroxyl, a carbonyl, a thiocarbonyl, an oxo, an alkoxy, an epoxy, a phosphoryl, a phosphate, a phosphonate, a phosphinate, an amino, an amido, an amidine, an imine, a cyano, a nitro, an azido, a thiol, an alkylthio, a sulfate, a sulfonate, a sulfamoyl, a sulfonamido, a sulfonyl, a cycloalkyl, a heterocyclyl, an aralkyl, or an aromatic or heteroaromatic or a Michael acceptor, wherein any two or more R^{a} and R^{a′} groups may be bonded together so as to form a ring system;
wherein X¹, X² and X³ are independently selected from
1) (CR^{a}R^{a′})ₓ, wherein x is selected from 1-20,
2) a heteroatom,
3) an alkenyl group of the formula: 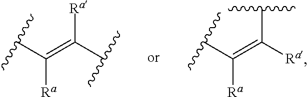
4) an alkynyl group of the formula: 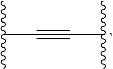 [SMILES: *C#C*]
5) a substituted or unsubstituted cycloalkyl group,
6) a substituted or unsubstituted aryl group,
7) a substituted or unsubstituted heterocyclic group,
8) a substituted or unsubstituted heteroaryl group,
9) an oligomeric group, and
wherein any of X¹, X² and X³ may be absent; and
wherein the monomer of Formula (3) has the structure: 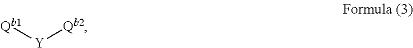
wherein Q^{b1} and Q^{b2} are nucleophilic functional groups and Y has a structure selected from: 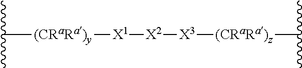
wherein [symbol] indicates a point of connection of Y to Q^{b1} or Q^{b2},
wherein y and z are in each case independently selected from zero or any integer,
wherein R^{a} and R^{a′} are in each case independently selected from hydrogen, an aliphatic group, a halogen, a hydroxyl, a carbonyl, a thiocarbonyl, an oxo, an alkoxy, an epoxy, a phosphoryl, a phosphate, a phosphonate, a phosphinate, an amino, an amido, an amidine, an imine, a cyano, a nitro, an azido, a thiol, an alkylthio, a sulfate, a sulfonate, a sulfamoyl, a sulfonamido, a sulfonyl, a cycloalkyl, a heterocyclyl, an aralkyl, or an aromatic or heteroaromatic or a Michael acceptor, wherein any two or more R^{a} and R^{a′} groups may be bonded together so as to form a ring system;
wherein X¹, X² and X³ are independently selected from
1) (CR^{a}R^{a′})ₓ, wherein x is selected from 1-20
2) a heteroatom,
3) an alkenyl group of the formula: 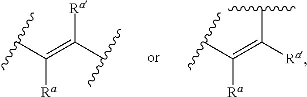
4) an alkynyl group of the formula: 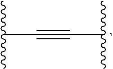 [SMILES: *C#C*]
5) a substituted or unsubstituted cycloalkyl group,
6) a substituted or unsubstituted aryl group,
7) a substituted or unsubstituted heterocyclic group,
8) a substituted or unsubstituted heteroaryl group,
9) an oligomeric group, and
wherein any of X¹, X² and X³ may be absent, and
wherein the compound of Formula (1) contains at least two reactive functional groups capable of participating in an observable dynamic equilibrium at a temperature of at least 40° C.

30. The dynamic network of claim 27 or 29, further comprising a Subunit C, wherein Subunit C is a compound containing at least one ester, amide or thioester group, or a mixture of compounds containing at least one ester, amide or thioester group.

31. The dynamic network of claim 27 or 29, further comprising one or more active substances.

32. A medical device comprising the dynamic network of claim 27 or 29.

33. The medical device of claim 32, further comprising:
a) a Subunit C, wherein Subunit C is defined above.
b) one or more active substances, or
c) a Subunit C and one or more active substances.

34. A dynamic network, wherein the network is prepared by reacting:
a) at least one compound of Formula (2) 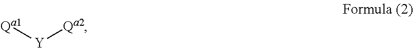
b) at least one compound of Formula (3) 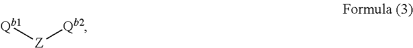
wherein Q^{a1} and Q^{a2} are each a moiety capable of reaction with Q^{b1} and Q^{b2}, and Y and Z independently have the following structure: 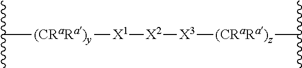
wherein [symbol] indicates a point of connection to Q^{a1}, Q^{a2}, Q_{b1}, or Q_{b2}
wherein y and z are in each case independently selected from zero or any integer,
wherein R^{a} and R^{a′} are in each case independently selected from hydrogen, an aliphatic group, a halogen, a hydroxyl, a carbonyl, a thiocarbonyl, an oxo, an alkoxy, an epoxy, a phosphoryl, a phosphate, a phosphonate, a phosphinate, an amino, an amido, an amidine, an imine, a cyano, a nitro, an azido, a thiol, an alkylthio, a sulfate, a sulfonate, a sulfamoyl, a sulfonamido, a sulfonyl, a cycloalkyl, a heterocyclyl, an aralkyl, or an aromatic or heteroaromatic or a Michael acceptor, wherein any two or more R^{a} and R^{a′} groups may be bonded together so as to form a ring system;
wherein X¹, X² and X³ are independently selected from
1) (CR^{a}R^{a′})ₓ, wherein x is selected from 1-20,
2) a heteroatom,
3) an alkenyl group of the formula: 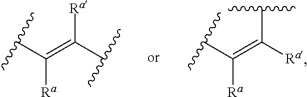
4) an alkynyl group of the formula: 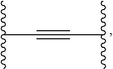 [SMILES: *C#C*]
5) a substituted or unsubstituted cycloalkyl group,
6) a substituted or unsubstituted aryl group,
7) a substituted or unsubstituted heterocyclic group,
8) a substituted or unsubstituted heteroaryl group,
9) an oligomeric group, and
wherein any of X¹, X² and X³ may also be absent, and
c) a catalyst, wherein the dynamic network contains at least two reactive functional groups capable of participating in an observable dynamic equilibrium at a temperature greater than 40° C.
